(19) United States
(12) Patent Application Publication
(10) Pub. No.: US 2010/0092477 A1
(43) Pub. Date: Apr. 15, 2010

(54) METHODS AND COMPOSITIONS FOR TREATING PROSTATE CANCER

(76) Inventors: Christopher Hovens, Surrey Hills (AU); Niall Corcoran, Flemington (AU); Anthony Costello, Port Melbourne (AU)

Correspondence Address: WINSTON & STRAWN LLP;PATENT DEPARTMENT, 1700 K STREET, N.W., WASHINGTON, DC, 20006, US

(21) Appl. No.: 12/568,590

(22) Filed: Sep. 28, 2009

Related U.S. Application Data
(63) Continuation-in-part of application No. PCT/AU08/00424, filed on Mar. 26, 2008.
(60) Provisional application No. 60/945,282, filed on Jun. 20, 2007.
(60) Provisional application No. 60/990,637, filed on Nov. 28, 2007.

(30) Foreign Application Priority Data
Mar. 27, 2007 (AU) 2007901628

(51) Int. Cl. A61K 39/395; C07K 14/00; C07K 16/00; A61P 5/24; C07H 21/04; C12N 15/63; A61P 35/04; A61K 38/16; A61K 31/7088

(52) U.S. Cl. 424/141.1; 530/387.3; 530/350; 530/388.1; 530/389.1; 536/23.4; 536/23.53; 435/320.1; 424/130.1; 514/12; 514/44 R

(57) ABSTRACT

A polypeptide comprising an androgen binding region, the androgen binding region capable of binding to an androgen at a sufficient affinity or avidity such that upon administration of the polypeptide to a mammalian subject the level of biologically available androgen is decreased. Specifically disclosed is an AR IgG1 Fc fusion protein, comprising the androgen binding domain of human androgen receptor and the Fc region of IgG. This fusion protein is used in the treatment of prostate cancer and testosterone flare.

### CROSS-REFERENCE TO RELATED APPLICATIONS

This application is a continuation-in-part application of international application PCT/AU2008/000424 filed Mar. 26, 2008 which claims priority to Australian application no. 2007901628 filed Mar. 27, 2007; International application PCT/AU2008/000424 also claims the benefit of U.S. provisional applications Nos. 60/945,282 filed Jun. 20, 2007 and 60/990,637 filed Nov. 28, 2007. The subject matter of these earlier filed applications is hereby incorporated by reference.

### FIELD OF THE INVENTION

The present invention relates generally to the field of oncology, and more particularly to the use of polypeptides and polypeptide complexes in the prevention or treatment of cancers of the prostate.

### BACKGROUND OF THE INVENTION

Prostate cancer is a disease causing significant morbidity and mortality throughout the world. The most prevalent form, prostatic adenocarcinoma, arises from the malignant transformation and clonal expansion of epithelial cells lining the secretory acini of the prostate gland. Cancers arising from other prostatic cells types, including transitional cell carcinoma, mesenchymal tumours and lymphomas are much less common.

Prostate adenocarcinoma is the most commonly diagnosed internal malignancy in men in North America, Northern and Western Europe, Australia and New Zealand, as well as parts of Africa. Over 650,000 new cases were diagnosed worldwide in the year 2002, with a mortality rate of over 30%. In Australia, 11,191 new cases were diagnosed in 2001 (age standardized incidence of 128.5 per 100,000) and 2,718 men died of the disease. The incidence is higher in the United States of America (173.8 per 100,000 per year) where in 2005 it is estimated there were over 230,000 new cases diagnosed, and over 30,000 deaths.

Given the prevalence and seriousness of the disease, significant research has been directed to achieving control or a cure for prostate cancer. There are a number of treatments known in the art, all of which have at least one adverse side effect.

Surgical removal of the prostate by radical prostatectomy with or without a regional lymph node dissection is the yardstick against which all other therapies are measured. The standard retropubic approach was repopularised in the 1980s and has been refined into a procedure with a high cure rate and low morbidity. With careful patient selection, 10 year biochemical free recurrence rates of 75% are reported. Improved understanding of pelvic anatomy, particularly at the prostatic apex and the course of the neurovascular bundles has reduced the two most common complications, incontinence and impotence, however these side effects remain significant problems.

External beam radiotherapy can achieve long-term survival in some patients, with success being proportional to the total dose delivered to the prostate tumour. In early series where median dose was limited due to rectal and urinary toxicity, biochemical failure occurred in over 50% of patients. Improvements in radiation planning and delivery such as using conformed or intensity-modulated protocols increase the precision by which the target volume corresponds to the tumour volume, allowing higher doses of radiotherapy to be delivered without an increase in complications. Modern series have a similar 10 year biochemical recurrence free survival to radical prostatectomy. The main difference is in the side effect profile, with radiotherapy being associated with a lower risk of urinary incontinence and impotence, at least in the short term, though potency rates do not differ greatly from those achieved with nerve sparing surgery. Severe toxicity such as chronic radiation cystitis or proctitis can be particularly difficult to manage if they occur.

Brachytherapy involves the placement of radioactive seeds transperineally directly into the prostate gland, and has reported biochemical-recurrence free survival rates similar to radical prostatectomy for highly selected cases. Two types of radioactivity sources are used, both of which have a short distance of action: low energy sources, typically iodine-125 or palladium-103 seeds which are placed permanently in the prostate, and high energy sources such as iridium-192 seeds which are placed temporarily. The main advantage of this technique over external beam radiotherapy is that with accurate preoperative computed tomography planning and appropriate seed placement under transrectal ultrasound control, a highly conformal dose distribution can be achieved which results in the delivery of much higher radiation doses with a lower incidence of rectal and neurovascular side-effects. One of the main difficulties even with modern practice is mismatch in dosimetry between planned implantation and the actual implantation because of seed migration, anisotropy of the individual seeds and inaccurate needle placement. In cases where inadequate dosimetry is suspected on postoperative imaging addition implants, or for high risk cases, adjuvant low dose external-beam radiotherapy may be added. The predominant complication is obstructive urinary symptoms due to gland oedema which may precipitate acute urinary retention. There is also a high risk of urinary incontinence following a formal transurethral resection.

Once cancerous cells have metastasized to areas remote from the prostate, removal of the gland becomes redundant. Despite the opportunity for early diagnosis with PSA testing, it is estimated that in the United States at least 14% of patients still present with disease that has spread outside the prostate gland and is no longer amenable to curative therapy. In addition, 30-40% of patients treated initially with curative intent will ultimately fail. Androgen deprivation therapy (ADT) is the usual first line treatment for patients with metastatic disease. Early randomised trials established that treatment of advanced prostate cancer with ADT improves symptoms, delays progression, and probably prolongs survival, with reported remission rates of 85-95%.

The growth of prostate cancer cells at some stages of disease can be reliant on the presence of androgen. Methods for altering the levels of androgen in the blood have been the subject of intensive investigation for many years, revealing a number of sites in the androgen endocrine axis that may be targeted, the most drastic method being bilateral orchidectomy, or surgical castration. For many years, this procedure was the ‘gold standard’ for achieving androgen deprivation. Following removal of the testes, serum testosterone falls rapidly to reach castrate levels (<50 ng/ml) within 9 hours. Side effects are secondary to this fall in testosterone and include hot flushes, reduced libido, fatigue and erectile dysfunction. Increasingly recognised are the medium to long term complications which include osteoporosis, weight gain, loss of muscle mass, anaemia, and a decline in cognitive function. Despite its relatively low cost, surgical castration has fallen from favour due to its irreversible nature and adverse psychological impact on the patient.

Androgen levels may be lowered using LHRH agonists and antagonists. These agents, including leuprolide, goserelin and triptorelin, are peptide analogues of LHRH, and are given as a subcutaneous depot injection every 1-4 months. When released in a pulsatile manner from the hypothalamus, LHRH stimulates the release of LH from the anterior pituitary, and thus testicular production of testosterone. Chronic administration of supra physiological levels however, after an initial increase in testosterone secretion, leads to downregulation of its cognate receptor and suppression of LH release. Castrate levels of testosterone are seen within 3 to 4 weeks. Because of the initial testosterone flare reaction, patients with critical tumour deposits must be covered with an antiandrogen when initially commencing a LHRH agonist. The side effects of treatment with LHRH agonists and antagonists are identical to those seen post bilateral orchidectomy.

Another class of drug are the antiandrogens. These agents compete with testosterone and dihydrotestosterone (DHT) for androgen receptor (AR) binding but do not themselves activate the receptor. Non-steroidal antiandrogens such as bicalutamide, flutamide and nilutamide act only at the level of the androgen receptor, including in the hypothalamus where testosterone inhibits LHRH secretion in a classical negative feedback loop. LH secretion, and thus serum testosterone, remains high, so the sexual side effects experienced with castration are reduced. However, due to the peripheral aromatization of testosterone to oestradiol, gynecomastia and breast pain are both common and troublesome. Steroidal antiandrogens, such as the progestin cyproterone acetate, also inhibit LH secretion, but are associated with the sexual side effects of surgical and medical castration. At least in metastatic disease, antiandrogen monotherapy has been shown to be inferior to castration and its use is therefore limited to patients unable or unwilling to tolerate the side effects of androgen suppression

Prolonged combination of an antiandrogen with an LHRH agonist is termed maximum androgen blockade as the regimen inhibits the effects of the remaining 5-10% of testosterone derived from the adrenal gland. Although an improvement in survival compared to castration alone is reported in some studies, routine use as a first line hormonal treatment is not recommended by most due to increased cost and side effect profile.

Estrogens are also known in the art for their ability to deplete androgen. Although initially the hormonal treatment of choice, diethylstilbestrol, which suppresses testosterone production by inhibiting the release of LHRH from the hypothalamus, is now rarely used as a first line agent because of concerns about cardiovascular toxicity.

Thus, the prior art describes many treatment modalities that either physically remove or destroy prostate cancer cells. Other approaches concentrate on limiting the amount of circulating testosterone by surgical or chemical means. From the foregoing description of the prior art, it is clear that every treatment has at least one problem, and may therefore be unsuitable for certain classes of patient. It is an aspect of the present invention to overcome or alleviate a problem of the prior art by providing alternative treatments for prostate cancer.

A reference herein to a patent document or other matter which is given as prior art is not to be taken as an admission that that document or matter was, in Australia, known or that the information it contains was part of the common general knowledge as at the priority date of any of the claims.

Throughout the description and claims of the specification, the word “comprise” and variations of the word, such as “comprising” and “comprises”, is not intended to exclude other additives, components, integers or steps.

### SUMMARY OF THE INVENTION

In one aspect, the present invention provides a polypeptide comprising an androgen binding region, the androgen binding region capable of binding to an androgen at a sufficient affinity or avidity such that upon administration of the polypeptide to a mammalian subject the level of biologically available androgen is decreased. Applicant proposes that the administration of a polypeptide capable of sequestering androgen (for example testosterone or dihydrotestosterone) in the body may have efficacy in the treatment of prostate cancer.

In the context of the invention, the level of biologically available androgen may be measured in the blood of the subject, or within a prostate cell, and especially a prostate epithelial cell. In one form of the invention the polypeptide is capable of decreasing the level of biologically available androgen such that the growth of a prostate cancer cell in the subject is decreased or substantially arrested.

The polypeptide may have an affinity for testosterone that is equal to or greater than the affinity between the androgen and a protein that naturally binds to testosterone such as the sex hormone binding globulin. The polypeptide may have an affinity for testosterone that is equal to or greater than the affinity between testosterone and the 5-alpha-reductase enzyme present in a prostate epithelial cell, or the androgen receptor present in a prostate epithelial cell.

In another form of the invention the polypeptide has an affinity for dihydrotestosterone that is equal to or greater than the affinity between dihydrotestosterone and the androgen receptor present in a prostate epithelial cell.

In one form of the polypeptide, the androgen binding region includes the androgen binding domain from the human androgen receptor, or the androgen binding domain from the sex hormone binding globulin.

In one form of the invention the polypeptide has a single androgen binding region. In another form, the polypeptide includes a carrier region such as the Fc region of human IgG. A further form of the polypeptide includes a multimerisation domain. The polypeptide may take the form of a fusion protein, a monoclonal antibody, a polyclonal antibody, or a single chain antibody.

The polypeptide may be capable of entering a prostate cell, and especially a prostate epithelial cell.

In another aspect, the present invention provides a nucleic acid molecule capable of encoding a polypeptide as described herein. A further aspect of the present invention provides a vector including a nucleic acid molecule as described herein.

In another aspect the present invention provides a composition comprising a polypeptide as described herein and a pharmaceutically acceptable carrier.

Yet a further aspect of the invention provides a method for treating or preventing prostate cancer in a subject, the method including administering to a subject in need thereof an effective amount of a ligand capable of binding androgen in the subject, such that the level of biologically available androgen in the subject is decreased. In one embodiment of the method, the ligand is a polypeptide as described herein.

Another aspect of the invention provides a method for treating or preventing prostate cancer, the method including administering to a subject in need thereof an effective amount of a nucleic acid molecule as described herein, or a vector as described herein.

In yet a further aspect, the present invention provides a method for treating or preventing testosterone flare including administering to a subject in need thereof an effective amount of a polypeptide as described herein.

Still a further aspect of the invention provides that use of a polypeptide as described herein in the manufacture of a medicament for the treatment or prevention of prostate cancer or testosterone flare.

In another aspect, the present invention provides the use of a nucleic acid molecule as described herein in the manufacture of a medicament for the treatment or prevention of prostate cancer or testosterone flare.

Still a further aspect provides the use of a vector as described herein in the manufacture of a medicament for the treatment or prevention of prostate cancer or testosterone flare.

### BRIEF DESCRIPTION OF THE FIGURES

FIG. 1 shows a map of pFUSE-hlgG1-Fc2.

FIG. 2 shows a map of pFUSE-hlgG1e2-Fc2.

FIG. 3 shows a map of pFUSE-mlgG1-Fc2.

FIG. 4 shows a Western blot of AR IgG1 Fc, and IgG1 Fc control fusion proteins.

FIG. 5 is a bar graph showing growth of human prostate cancer cell line LNCaP in the presence of various media and treatments over 5 days as assessed by calcein fluorescence assay.

FIG. 6A is a graph depicting standard curve of known free testosterone concentrations versus free testosterone concentration of control mouse serum and free testosterone concentration of serum from mice injected with the AR-IgG1 Fc fusion protein.

FIG. 6B is a bar graph showing mean values of free testosterone levels in serum of mice either injected or not with AR IgG Fc fusion protein (25 ng).

FIG. 6C is a bar graph showing average values of free testosterone levels in serum of SCID/NOD mice either injected with AR-LBD IgG1 Fc fusion protein (200 μl of 1 ng/μl) or with control IgG1 Fc protein (200 μl of 1 ng/μl).

FIG. 6D is a bar graph showing average percentage values of free testosterone levels in serum of SCID/NOD mice either injected with AR-LBD IgG1 Fc fusion protein (200 μl of 1 ng/μl) or with control IgG1 Fc protein (2Q0 μl of 1 ng/μl).

FIG. 7A depicts representative images of final prostate tumour sizes of NUDE mice either injected twice with either a control IgG1 Fc protein or AR-LBD IgG1 Fc fusion protein.

FIG. 7B is a graphical depiction of prostate tumour volumes throughout time course of the experiment of male NUDE mice either injected twice with either control IgG1 Fc protein or with AR-LBD IgG1 Fc fusion protein.

FIG. 7C is a graphical depiction of final prostate tumour weights (mg) of male NUDE mice either injected twice with either control IgG1 Fc protein (IgG) or with AR-LBD IgG1 Fc fusion protein (AR).

FIG. 8 is a graphical depiction of the measured levels of human PSA secreted into the culture media of human prostate cancer cell line LNCaP (ATCC) in the presence of various treatments, including 20 ng/ml AR-IgG Fc polypeptide, as depicted over a 5 day period.

FIG. 9 is a graphical depiction of the measured levels of human PSA secreted into the culture media of human prostate cancer cell line LNCaP (ATCC) in the presence of various treatments, including 20 ng/ml AR-IgG Fc polypeptide, as depicted over a 5 day period.

FIG. 10 is a graphical depiction of the measured levels of human PSA secreted into the blood of SCIDINOD mice injected into the prostate with the human prostate cancer cell line LNCaP (ATCC).

### DETAILED DESCRIPTION OF THE INVENTION

In a first aspect the present invention provides a polypeptide comprising an androgen binding region, the androgen binding region capable of binding to an androgen at a sufficient affinity or avidity such that upon administration of the polypeptide to a mammalian subject the level of biologically available androgen is decreased. Applicant proposes that polypeptides having the ability to bind to an androgen are useful in decreasing the level of hormones such as testosterone and dihydrotestosterone that are biologically available to stimulate the androgen receptor in prostate cancer cells. In the normal course of events, the androgen receptor binds testosterone or its active metabolite dihydrotestosterone. After dissociation of heat shock proteins the receptor enters the nucleus via an intrinsic nuclear localization signal. Upon steroid hormone binding, which may occur either in the cytoplasm or in the nucleus, the androgen receptor binds as homodimer to specific DNA elements present as enhancers in upstream promoter sequences of androgen target genes. The next step is recruitment of coactivators, which can form the communication bridge between receptor and several components of the transcription machinery. The direct and indirect communication of the androgen receptor complex with several components of the transcription machinery such as RNA polymerase II, TATA box binding protein (TBP), TBP associating factors, and general transcription factors, are key events in nuclear signaling. This communication subsequently triggers mRNA synthesis and consequently protein synthesis, which finally results in an androgen response.

Activation of the androgen receptor in prostate epithelial cells stimulates cell proliferation by increasing the transcription of genes encoding proteins such as cdks 2 and 4 that drive progression through G1, ultimately leading to Rb hypophosphorylation and commitment to cell division. Androgen receptor activation has recently been shown to result in non-genomic activation of a number of mitogenic cascades, including src/raf/ERK and PI3K/AKT. Activation of these pathways occurs rapidly, is ligand dependent, and results from direct interaction between the receptor and upstream kinases. While this stimulation of cell proliferation is necessary to maintain homeostasis in the prostate (1-2% of luminal secretory cells are lost per week though attrition or injury) the growth response must be regulated to prevent the uncontrolled growth seen in the cancerous prostate. The polypeptides described herein are proposed to limit or prevent activation of the androgen receptor by androgen, thereby decreasing or substantially arresting proliferation of prostate cells.

The present invention is distinct from approaches of the prior art that aim to decrease the production of testosterone. As discussed in the Background section herein, this has been achieved by removal of the testes, or decreasing the production of testosterone by the testes using compounds such as GnRH/LHRH agonists, GnRH antagonists, and cyproterone acetate (CPA). Compounds such as ketoconazole and corticosteroids have been used in the prior art to decrease the production of testosterone precursors by the adrenal glands. By contrast, the polypeptides of the present invention do not directly interfere with the production of androgen by the testes or adrenal glands.

The present invention is also distinguished from prior art treatments that act to block 5-alpha-reductase, the enzyme present in prostate cells that converts testosterone to dihydrotestosterone. While both testosterone and dihydrotestosterone are able to bind the androgen receptor, dihydrotestosterone is the more potent ligand. Thus, while compounds such as finasteride and dutasteride can limit the level of dihydrotestosterone in a prostate cell, they are unable to affect the binding of testosterone directly to the androgen receptor. In one embodiment of the invention, the polypeptides of the present invention are proposed to bind both testosterone and dihydrotestosterone, thereby overcoming the problems of 5-alpha-reductase inhibitors.

The polypeptides of the present invention are also different to compounds of the prior art such as CPA, bicalutamide, nilutamide and flutamide that bind to the androgen receptor. While these compounds have some efficacy in blocking the receptor they are incapable (as a monotherapy) to sufficiently limit androgen signaling. As mentioned supra antiandrogen monotherapy has been demonstrated to be inferior to castration at prolonging survival in metastatic disease. In addition, about 10% of hormone refractory prostate cancer patients have one or more mutations in the androgen receptor gene such that compounds of the prior art may act as partial agonists of the androgen receptor.

By contrast, the polypeptides of the present invention bind to molecules that have a set chemical structure, and “escape” variants do not need to be accounted for.

In one form of the invention the polypeptide is capable of binding to testosterone present in the blood. The vast majority of testosterone in the blood is bound to proteins such as steroid hormone binding globulin (SHBG) and albumin. The remaining testosterone (only about 1-2%) is biologically available. It is this unbound or “free” testosterone that is available for activating the androgen receptor in prostate cells.

In another form of the invention the polypeptide is capable of entering a prostate cell, and particularly a prostate epithelial cell. As used herein, the term “prostate cell” is intended to include a cell within or associated with the actual prostate gland, or a cell that has metastasized from the gland and has lodged in a remote location to form a secondary tumour. The term is also intended to include a cell that is in transit from the prostate gland to the final site of lodgement at the secondary tumour. The advantage of a polypeptide capable of entering the cell is that the opportunity is increased to bind all testosterone and/or dihydrotestosterone. It is pertinent to note that although after androgen ablation therapy serum testosterone levels decrease by >90%, the concentration of dihydrotestosterone in the prostate declines by only 60% (Labrie, F et al., Treatment of prostate cancer with gonadotropin releasing hormone agonists. Endocr review, 19t36. 7(1): 67-74). This failure to achieve more complete ablation of androgen in the prostate may be due to cells in the organ retaining a reservoir of androgen capable of acting in an autocrine manner. There is also evidence to suggest that hormone refractory prostate cancer cells are capable of synthesizing androgens from circulating precursor molecules. Given that androgen receptor blockers of the prior art are simple competitive inhibitors, it is likely that intraprostatic steroidogenesis leads to locally increased concentrations of androgens thereby contributing at least in part to the failure of these therapies. By directly targeting intracellular androgen, Applicants propose a more complete ablation of androgen is possible using the polypeptides described herein. Certain forms of the polypeptide including features that facilitate entry into prostate cells are disclosed infra.

In a further form of the invention the polypeptide is capable of binding to androgen present in both the blood and in cells of the prostate. Typically, a polypeptide that has the ability to enter a cell, will also be operable in the blood.

It is proposed that the polypeptide is capable of removing testosterone such that the level of androgen available to bind to its receptor is decreased such that the growth of a prostate cancer cell in the subject is decreased or substantially arrested.

Typically, the polypeptide has an affinity or avidity for androgen that is sufficiently high such that upon administration of the polypeptide to a mammalian subject, the polypeptide is capable of decreasing biologically available androgen in the blood or prostate cell of the subject to a level lower than that demonstrated in the subject prior to administration of the polypeptide. As used herein, the term “biologically available androgen” means androgen that is capable of exerting its biological activity. As will be understood, the present invention is directed to polypeptides that are capable of decreasing the level of androgen available to bind to an androgen receptor in a prostate cell of the subject. Thus, in the context of the present invention where the androgen is testosterone, the term “biologically available” means that the testosterone is free for conversion to dihydrotestosterone, which subsequently binds to the androgen receptor. Where the androgen is dihydrotestosterone (typically located intracellularly) the term “biologically available” means that the dihydrotestosterone is free to bind to an androgen receptor.

The vast majority of testosterone circulating in the blood is not biologically available in that about 98% is bound to serum protein. In men, approximately 40% of serum protein bound testosterone is associated with sex hormone binding globulin (SHBG), which has an association constant (Ka) of about 1×10⁹ L/mol. The remaining approximately 60% is hound weakly to albumin with a Ka of about 3×10⁴ L/mol.

As discussed supra, the polypeptide is capable of decreasing biologically available androgen. In this regard, androgen assays that measure levels of total testosterone in the blood (i.e. free testosterone in addition to bound testosterone) may not be relevant to an assessment of whether a polypeptide is capable of decreasing biologically available androgen. A more relevant assay would be one that measures free testosterone. These assays require determination of the percentage of unbound testosterone by a dialysis procedure, estimation of total testosterone, and the calculation of free testosterone. Free testosterone can also be calculated if total testosterone, SHBG, and albumin concentrations are known (Sødergard et al, Calculation of free and bound fractions of testosterone and estradiol-17β to human plasma proteins at body temperature. J Steroid Biochem. 1(3:801-810; the contents of which is herein incorporated by reference). Methods are also available for determination of free testosterone without dialysis. These measurements may be less accurate than those including a dialysis step, especially when the testosterone levels are low and SHBG levels are elevated (Rosner W. 1997 Errors in measurement of plasma free testosterone. J Clin Endocrinol Metabol. 82:2014-2015; the contents of which is herein incorporated by reference, Giraudi et al. 1988. Effect of tracer binding to serum proteins on the reliability of a direct free testosterone assay. Steroids. 52:423-424; the contents of which is herein incorporated by reference). However, these assays may nevertheless be capable of determining whether or not a polypeptide is capable of decreasing biologically available testosterone.

Another method of measuring biologically available testosterone is disclosed by Nankin et al 1986 (Decreased bioavailable testosterone in aging normal and impotent men. J Clin Endocrinol Metab. 63:1418-1423; the contents of which is herein incorporated by reference. This method determines the amount of testosterone not bound to SHBG and includes that which is nonprotein bound and weakly bound to albumin. The assay method relies on the fact SHBG is precipitated by a lower concentration of ammonium sulfate, 50%, than albumin. Thus by precipitating a serum sample with 50% ammonium sulfate and measuring the testosterone value in the supernate, non-SHBG bound or biologically available testosterone is measured. This fraction of testosterone can also be calculated if total testosterone, SHBG, and albumin levels are known.

Further exemplary methods of determining levels of biologically available testosterone are disclosed in de Ronde et al., 2006 (Calculation of bioavailable and free testosterone in men: a comparison of 5 published algorithms. Clin Chem 52(9): 1777-1784; the contents of which is herein incorporated by reference).

In determining whether or not a polypeptide is capable of decreasing biologically available androgen, the skilled person will understand that it may be necessary to account for the natural variability of androgen levels that occur in an individual. It is known that androgen levels fluctuate in an individual according to many factors, including the time of day and the amount of exercise performed. For example, it is typically observed that testosterone levels are higher in the morning as compared with a sample taken in the evening. Even in consideration of these variables, by careful planning of sample withdrawal, or by adjusting a measurement obtained from the individual, it will be possible to ascertain whether the level of biologically available androgen in an individual (and the resultant effect on prostate cancer growth) has been affected by the administration of a polypeptide as described herein.

In one form of the invention the polypeptide has an affinity or avidity for androgen that is equal to or greater than that noted for natural carriers of androgen in the body. As discussed supra, natural carriers in the blood include SHBG and serum albumin. It will be appreciated that the binding of testosterone to these natural carriers is reversible, and an equilibrium exists between the bound and unbound form of testosterone. In one form of the invention, to decrease the level of biologically available testosterone to below that normally present (i.e. less than 1-2%) the polypeptide has an affinity or avidity for testosterone that is greater than that between SHBG and testosterone, or albumin and testosterone. Thus in one embodiment of the invention, the polypeptide has an association constant for testosterone that is greater than that for a natural carrier of testosterone such as SHBG or albumin.

In another form of the invention the polypeptide has an association constant for testosterone that is about equal or less than that for a natural carrier of testosterone such as SHBG or albumin. In this embodiment, while free testosterone may bind to SHBG or albumin in preference to the polypeptide, addition of polypeptide to the circulation may still be capable of decreasing the level of biologically available testosterone. Where the polypeptide has a low affinity or avidity for androgen, it may be necessary to administer the polypeptide in larger amounts to ensure that the level of androgen is sufficiently depleted.

In another form of the invention the polypeptide has an affinity or avidity for testosterone that is sufficiently high such that it is capable of maintaining decreased levels of testosterone levels within a prostate cell, and more particularly a prostate epithelial cell. Administration of the polypeptide can achieve this result by depleting the level of testosterone in the circulation such that little or no testosterone can therefore enter the prostate cell. Additionally, or alternatively, the polypeptide is capable of entering the prostate cell and binding to intracellular testosterone and or dihydrotestosterone.

Given that testosterone is converted into dihydrotestosterone in cells of the prostate, another form of the invention provides that the polypeptide has an affinity or avidity for dihydrotestosterone that is sufficiently high such that it is capable of maintaining decreased levels of dihydrotestosterone levels within a prostate cell. These forms of the polypeptide interfere with the binding of testosterone and/or dihydrotestosterone to the androgen receptor within the prostate cell. Testosterone and dihydrotestosterone are capable of binding to common targets (for example, the androgen receptor) and it is therefore proposed that the polypeptides described herein are capable of binding to both testosterone and dihydrotestosterone. As discussed supra the proliferation of cancerous prostate cells may be decreased or arrested by inhibiting the androgen response of the cells.

In a further form of the invention the polypeptide has an affinity or avidity for testosterone that is equal to or greater than that between testosterone and the 5-alpha-reductase enzyme present in prostate cells. As discussed supra upon entry of testosterone into the prostate cell, the steroid is typically converted to dihydrotestosterone by the enzyme 5-alpha-reductase. In order to decrease the opportunity for intracellular testosterone to associate with the enzyme the polypeptide has a greater affinity than the enzyme for testosterone. By virtue of the superior binding of testosterone with the polypeptide, the opportunity for conversion of testosterone to dihydrotestosterone is limited. However, given the potential for a reversible association of testosterone with the polypeptide, all testosterone may eventually be converted to the dihydro form. In that case it is desirable for the polypeptide to be capable of binding to testosterone and dihydrotestosterone, or for two polypeptide species to be used (one for binding testosterone, and the other for binding dihydrotestosterone). In this embodiment of the invention, the precursor and product of the 5-alpha-reductase catalyzed reaction are liable to be bound to polypeptide the end result being lowered concentrations of both molecules available for binding to the androgen receptor.

In a further embodiment, the polypeptide has an affinity or avidity for dihydrotestosterone that is equal to or greater than the affinity or avidity of the androgen receptor for dihydrotestosterone. In another embodiment, the polypeptide has an affinity or avidity for testosterone that is equal to or greater than the affinity or avidity of the androgen receptor for testosterone.

In one form of the invention the androgen binding region of the polypeptide includes a sequence or sequences derived from human androgen receptor. The gene encoding the receptor is more than 90 kb long and codes far a protein that has 3 major functional domains. The N-terminal domain, which serves a modulatory function, is encoded by exon 1 (1,586 bp). The DNA-binding domain is encoded by exons 2 and 3 (152 and 117 bp, respectively). The steroid-binding domain is encoded by 5 exons which vary from 131 to 288 hp in size. The amino acid sequence of the human androgen receptor protein is described by the following sequence (SEQ ID NO: 1).

mevqlglgrv yprppsktyr gafgnltqsv reviqnpgpr
hpeaasaapp gasllllqqq qqqqqqqqqq qqqqqqqqet
sprqqqqqqg edgspqahrr gptgylvlde eqqpsqpqsa
lechpergcv pepgaavaas kglpqqlpap pdeddsaaps
tlsllgptfp glsscsadlk dilseastmq llqqqqqeav
segsssgrar easgaptssk dnylggtsti sdnakelcka
vsvsmglgve alehlspgeq lrgdcmyapl lgvppavrpt
pcaplaeckg sllddsagks tedtaeyspf kggytkgleg
eslgcsgsaa agssgtlelp stlslyksga ldeaaayqsr
dyynfplala gpppppppph phariklenp ldygsawaaa
aaqcrygdla slhgagaagp gsgspsaaas sswhtlftae
egqlygpcgg gggggggggg gggggggggg ggeagavapy
gytrppqgla gqesdftapd vwypggmvsr vpypsptcvk
semgpwmdsy sgpygdmrle tardhvlpid yyfppqktcl
icgdeasgch ygaltcgsck vffkraaegk qkylcasrnd
ctidkfrrkn cpscrlrkcy eagmtlgark lkklgnlklq
eegeasstts pteettqklt vshiegyecq piflnvleai
epgvvcaghd nnqpdsfaal lsslnelger qlvhvvkwak
alpgfrnlhv ddqmaviqys wmglmvfamg wrsftnvnsr
mlyfapdlvf neyrmhksrm ysqcvrmrhl sqefgwlqit
pqeflcmkal llfsiipvdg lknqkffdel rmnyikeldr
iiackrknpt scsrrfyqlt klldsvqpia relhqftfdl
likshmvsvd fpemmaeiis vqvpkilsgk vkpiyfhtq

The present invention also includes functional equivalents of sequences as described herein. As will be understood, bases or amino acid residues may be substituted, repeated, deleted or added without substantially affecting the biological activity of the polypeptide. It will therefore be understood that strict congruence with the above sequence is not necessarily required.

In one embodiment, the androgen binding region includes or consists of the steroid binding domain of the human androgen receptor, but is devoid of regions of the receptor that are not involved in steroid binding. The identity of the steroid binding domain of the androgen receptor has been the subject of considerable research (Ai et al, Chem Res Toxicol 2003, 16, 1652-1660; Bohl et al, J Biol Chem 2005, 280(45) 37747-37754; Duff and McKewan. Mol Endocrinol 2005, 19(12) 2943-2954; Ong et al, Mol Human Reprod 2002, 8(2) 101-108; Poujol et al, J Biol Chem 2000, 275(31) 24022-24031; Rosa et al, J Clin Endocrinol Metab 87(9) 4378-4382; Marhefka et al, J Med Chem 2001, 44, 1729-1740; Matias et al, J Biol Chem 2000, 275(34) 26164-26171, McDonald et al, Cancer Res 2000, 60, 2317-2322; Sack et al, PNAS 2001, 98(9) 4904-4909; Steketee et al, Int J Cancer 2002, 100, 309-317, the contents of all aforementioned publications are herein incorporated by reference). While the exact residues essential for steroid binding are not known, it is generally accepted that the region spanning the approximately 250 amino acid residues in the C-terminal end of the molecule is involved (Trapman et al (1988). Biochem Biophys Res Commun 153, 241-248, the contents of which is herein incorporated by reference).

In one embodiment of the invention the androgen binding region includes or consists of the sequence defined by the 230 C-terminal amino acids of SEQ ID NO:1 (i.e. the sequence dnnqpd . . . iyfhtq).

Some studies have considered the crystal structure of the steroid binding domain of the human androgen receptor in complex with a synthetic steroid. For example, Sack et al (ibid) propose that the 3-dimensional structure of the receptor includes a typical nuclear receptor ligand binding domain fold. Another study proposes that the steroid binding pocket has been consists of 18 (noncontiguous) amino acid residues that interact with the ligand (Matiae et al, ibid). It is emphasized that this study utilized a synthetic steroid ligand (R1881) rather than actual dihydrotestosterone. The binding pocket for dihydrotestosterone may include the same residues as that shown for R1181 or different residues.

Further crystallographic data on the steroid binding domain complexed with agonist predict 11 helices (no helix 2) with two anti-parallel β-sheets arranged in a so-called helical sandwich pattern. In the against-bound conformation the carboxy-terminal helix 12 is positioned in an orientation allowing a closure of tile steroid binding pocket. The fold of the ligand binding domain upon hormone binding results in a globular structure with an interaction surface for binding of interacting proteins like co-activators.

From the above, it will be understood that the identity of the minimum residues required for binding androgen has not been settled at the filing date of this application. Accordingly, the present invention is not limited to polypeptides including any specific region of the androgen receptor as discussed supra. It is therefore to be understood that the scope of the present invention is not necessarily limited to any specific residues as detailed herein.

In any event, while the steroid binding domain of the androgen receptor is generally well conserved, the skilled person understands that various alterations may be made without completely ablating the ability of the sequence to bind steroid. Indeed it may be possible to alter the sequence to improve the ability of the domain to bind androgen. Therefore, the scope of the invention extends to functional derivatives of the steroid binding domain of the androgen receptor. It is expected that certain alterations could be made to the ligand binding domain sequence of the androgen receptor without substantially affecting the ability of the domain to bind androgen. For example, the possibility exists that certain amino acid residues may be deleted, substituted, or repeated. Furthermore, the sequence may be truncated at the C-terminus and/or the N-terminus. Furthermore additional bases may be introduced within the sequence. Indeed, it may be possible to achieve a sequence having an increased affinity for androgen by trialing a number of alterations to the amino acid sequence. The skilled person will be able to ascertain the effect (either positive or negative) on the binding by way of standard association assay with androgen, as described supra.

In one form of the invention the androgen binding region of the polypeptide includes a sequence or sequences derived from the steroid binding domain of the human sex hormone binding protein. The sequence of human SHBG is described by the following sequence (SEQ ID NO: 2)

esrgplatsr llllllllll rhtrqgwalr pvlptqsahd
ppavhlsngp gqepiavmtf dltkitktss sfevrtwdpe
gvifygdtnp kddwfmlglr dgrpeiqlhn hwaqltvgag
prlddgrwhq vevkmegdsv llevdgeevl rlrqvsgplt
skrhpimria lggllfpasn lrlplvpald gclrrdswld
kqaeisasap tslrscdves npgiflppgt qaefnlrdip
qphaepwafs ldlglkqaag sghllalgtp enpswlslhl
qdqkvvlssg sgpgldlplv lglplqlkls msrvvlsqgs
kmkalalppl glapllnlwa kpqgrlflga lpgedsstsf
clnglwaqgq rldvdqalnr sheiwthscp qspgngtdas
h

The scope of the invention extends to fragments and functional equivalents of the above protein sequence.

As discussed supra, SHBG is responsible for binding the vast majority of testosterone in the serum. Accordingly, in one embodiment of the invention the steroid binding domain of the polypeptide includes the testosterone binding domain of SHBG. This domain comprises the region defined approximately by amino acid residues 1β to 177.

While the polypeptide may have more than one androgen binding region, in one form of the invention the polypeptide has only a single androgen binding region. This form of the polypeptide may be advantageous due to the potentially small size of the molecule. A smaller polypeptide may have a longer half life in the circulation, or may elicit a lower level of immune response in the body. A smaller polypeptide may also have a greater ability to enter a prostate cell to neutralize intracellular androgen.

It is emphasized that the steroid binding region of the polypeptide is not restricted to any specific sequence or sequences described herein. The domain may be determined by reference to any other molecule (natural or synthetic) capable of binding androgen including any carrier protein, enzyme, receptor, or antibody.

In one form of the invention, the polypeptide includes a carrier region. The role of the carrier region is to perform any one or more of the following functions: to generally improve a pharmacological property of the polypeptide including bioavailability, toxicity, and half life; limit rejection or destruction by an immune response; facilitate the expression or purification of the polypeptide when produced in recombinant form; all as compared with a polypeptide that does not include a carrier region.

In one form of the invention, the carrier region comprises sequence(s) of the Fc region of an IgG molecule. Methods are known in the art for generating Fc-fusion proteins, with a number being available in kit form by companies such as Invivogen (San Diego Calif.). The Invivogen system is based on the pFUSE-Fc range of vectors which include a collection of expression plasmids designed to facilitate the construction of Fe-fusion proteins. The plasmids include wild-type Fc regions from various species and isotypes as they display distinct properties

The plasmids include sequences from human wild type Fc regions of IgG1, IgG2, IgG3 and IgG4. Furthermore, engineered human Fc regions are available that exhibit altered properties.

pFUSE-Fc plasmids feature a backbone with two unique promoters: EF1 prom/HTLV 5′UTR driving the Fc fusion and CMV enh/FerL prom driving the selectable marker Zeocin. The plasmid may also contain an IL2 signal sequence for the generation of Fc-Fusions derived from proteins that are not naturally secreted.

The Fc region binds to the salvage receptor FcRn which protects the fusion protein from lysosomal degradation giving increased half-life in the circulatory system. For example, the serum half-life of a fusion protein including the human IgG3 Fc region is around one week. In another form of the invention the Fc region includes human IgG1, IgG2 or IgG4 sequence which increases the serum half-life to around 3 weeks. Serum half-life and effector functions (if desired) can be modulated by engineering the Fc region to increase or reduce its binding to FcRn, FcyRs and C1q respectively.

Increasing the serum persistence of a therapeutic antibody is one way to improve efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton P R. et al., 2004. Engineered human IgG antibodies with longer serum half-lives in primates. J Biol. Chem. 279(8):6213-6; the contents of which is herein incorporated by reference, Vaccaro C. et al., 2005. Engineering the Fc region of immunoglobulin G to modulate in vivo antibody levels. Nat. Biotechnol. 23(10): 1283-8; the contents of which is herein incorporated by reference).

In one form of the invention, the carrier region comprises sequence(s) of the wild type human Fc IgGI region, as described by the following sequence (SEQ ID NO: 3), or functional equivalents thereof

thtcppcpap ellggpsvfl fppkpkdtlm isrtpevtcv
vvdvshedpq vkfnwyvdgv qvhnaktkpr eqqynstyrv
vsvltvlhqn wldgkeykck vsnkalpapi ektiskakgq
prepqvytlp psreemtknq vsltclvkgf ypsdiavewe
sngqpennyk ttppvldsdg sfflyskltv dksrwqqgnv
fscsvmheal hnhytqksls lspg

While the polypeptide may be a fusion protein such as that described supra, it will be appreciated that the polypeptide may take any form that is capable of achieving the aim of binding an androgen such that the level of androgen in the blood or prostate cell is decreased.

For example, the polypeptide may be a therapeutic antibody. Many methods are available to the skilled artisan to design therapeutic antibodies that are capable of binding to a predetermined target, persist in the circulation for a sufficient period of time, and cause minimal adverse reaction on the part of the host (Carter, Nature Reviews (Immunology) Volume 6, 2006; the contents of which is herein incorporated by reference).

In one embodiment, the therapeutic antibody is a single clone of a specific antibody that is produced from a cell line, including a hybridoma cell. There are four classifications of therapeutic antibodies: murine antibodies; chimeric antibodies; humanized antibodies; and fully human antibodies. These different types of antibodies are distinguishable by the percentage of mouse to human parts making up the antibodies. A murine antibody contains 100% mouse sequence, a chimeric antibody contains approximately 30% mouse sequence, and humanized and fully human antibodies contain only 5-10% mouse residues.

Fully murine antibodies have been approved for human use on transplant rejection and colorectal cancer. However, these antibodies are seen by the human immune system as foreign and may need further engineering to be acceptable as a therapeutic.

Chimeric antibodies are a genetically engineered fusion of parts of a mouse antibody with parts of a human antibody. Generally, chimeric antibodies contain approximately 33% mouse protein and 67% human protein. They combine the specificity of the murine antibody with the efficient human immune system interaction of a human antibody. Chimeric antibodies can trigger an immune response and may require further engineering before use as a therapeutic. In one form of the invention, the polypeptides include approximately 67% human protein sequences.

Humanized antibodies are genetically engineered such that the minimum mouse part from a murine antibody is transplanted onto a human antibody. Typically, humanized antibodies are 5-10% mouse and 90-95% human. Humanized antibodies counter adverse immune responses seen in murine and chimeric antibodies. Data from marketed humanized antibodies and those in clinical trials show that humanized antibodies exhibit minimal or no response of the human immune system against them. Examples of humanized antibodies include Enbrel® and Remicade®. In one form of the invention, the polypeptides are based on the non-ligand specific sequences included in the Enbrel© or Remicade© antibodies.

Fully human antibodies are derived from transgenic mice carrying human antibody genes or from human cells. An example of this is the Humira© antibody. In one form of the invention, the polypeptide of the present invention is based on the non-ligand specific sequences included in the Humira© antibody.

The polypeptide may be a single chain antibody (scFv), which is an engineered antibody derivative that includes heavy and light chain variable regions joined by a peptide linker. ScFv antibody fragments are potentially more effective than unmodified IgG antibodies. The reduced size of 27-30 kDa allows penetration of tissues and solid tumors more readily (Huston et al. (1993). Int. Rev. Immunol. 10, 195-217; the contents of which is herein incorporated by reference). Methods are known in the art for producing and screening scFv libraries for activity, with exemplary methods being disclosed in is disclosed by Walter et al 2001, High-throughput screening of surface displayed gene products Comb Chem High Throughput Screen; 4(2): 193-205; the contents of which is herein incorporated by reference.

The polypeptide may have greater efficacy as a therapeutic if in the form of a multimer. The polypeptide may be effective, or have improved efficacy when present as a homodimer, homotrimer, or homotetramer; or as a heterodimer heterotrimer, or heterotetramer. In these cases, the polypeptide may require multimerisation sequences to facilitate the correct association of the monomeric units. Thus, in one embodiment the polypeptide includes a multimerisation region. It is anticipated that where the steroid binding region of the polypeptide includes sequences from SHBG, a multimerisation region may be included.

In another aspect, the present invention, provides a composition comprising a polypeptide of the present invention in combination with a pharmaceutically acceptable carrier. The skilled person will be enabled to select the appropriate carrier(s) to include in the composition. Potentially suitable carriers include a diluent, adjuvant, excipient, or vehicle with which the polypeptide is administered. Diluents include sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The polypeptides of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethyl amino ethanol, histidine, procaine, etc.

Furthermore, aqueous compositions useful for practicing the methods of the invention have physiologically compatible pH and osmolality. One or more physiologically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in a physiologically acceptable range. One or more physiologically acceptable salts can be included in the composition in an amount sufficient to bring osmolality of the composition into an acceptable range. Such salts include those having sodium, potassium or ammonium cations and chloride, citrate, ascorbate, borate, phosphate, bicarbonate, sulfate, thiosulfate or bisulfite anions.

In another aspect, the present invention includes a method for treating or preventing prostate cancer in a subject, the method comprising administering to a subject in need thereof an effective amount of a ligand capable of binding androgen in the subject, such that the level of biologically available androgen in the subject is decreased. In one form of the method, the ligand is a polypeptide as described herein.

The amount of the polypeptide that will be effective for its intended therapeutic use can be determined by standard clinical techniques well known to clinicians. Generally, suitable dosage ranges for intravenous administration are generally about 20 to 500 micrograms of active compound per kilogram body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The dosage regime could be arrived at by routine experimentation on the part of the clinician. Generally, the aim of therapy would be to bind all, or the majority of free androgen in the blood and prostate cell to the polypeptide. In deciding an effective dose, the amount of polypeptide could be titrated from a low level up to a level whereby the level of biologically available testosterone is undetectable. Methods of assaying biologically available testosterone are known in the art, as discussed elsewhere herein. Alternatively, it may be possible to theoretically estimate (for example on a molar basis) the amount of polypeptide required to neutralize substantially all free testosterone. Alternatively, the amount could be ascertained empirically by performing a trial comparing the dosage with clinical effect. This may give an indicative mg/kg body weight dosage for successful therapy.

The duration of treatment and regularity of dosage could also be arrived at by theoretical methods, or by reference to the levels of biologically available testosterone in the patient and/or clinical effect.

In one form of the method, the level of biologically available androgen is measured in the blood of the subject, and/or in a prostate cell (and particularly a prostate epithelial cell) of the subject.

The methods of treatment will be most efficacious where the prostate cancer is in the androgen dependent phase. However, it will be appreciated that the polypeptides may be used prophylactically before the prostate cancer has been diagnosed. Polypeptide may be administered in this way to a person with a strong family history of prostate cancer, or with any other predisposition to the disease.

It is contemplated that the methods of treatment and prophylaxis included the use a polypeptide as described herein as a monotherapy, or in combination with at least one other therapeutic used in the treatment of prophylaxis of prostate cancer. It is proposed that in some forms of the invention use of the polypeptides as described herein as part of a combination therapy provides advantages. An advantage may be due to the unique mechanism by which the polypeptides of the present invention act as therapeutics. As discussed herein, the polypeptides act to bind androgen, such that the level of biologically available androgen in the blood and/or prostate cell is decreased. This is distinct from prior art therapeutics that typically act by decreasing the amount of androgen secreted by the body. It is therefore proposed that by the use of combination, and additive or synergistic effect may be realized.

As a non-limiting example of a combination therapy, an androgen agonist and a polypeptide of the present invention may be co-administered to patients in the early androgen dependent phase of the disease. Androgen agonist drugs (such as leuprolide) are typically administered with the aim of inducing castrate levels of androgens in the blood. This is typically defined as a 90% reduction in levels of serum testosterone. However, it is contemplated that an advantage is gained where low levels of androgen agonist drugs are administered such that serum testosterone is reduced to supra-castrate levels (for example, a reduction of from about 25% to about 75%). In this case, the polypeptide is administered with the aim of neutralizing the remaining testosterone. The advantage of this approach, is that for a given dose of polypeptide a longer half-life results since the polypeptide would not have neutralize all of the serum testosterone but only 25 to 50% of normal levels.

Combination treatment including a polypeptide of the present invention will further decrease the levels of serum testosterone by physically sequestering the remaining testosterone. In this example, the different, yet complementary mechanisms of action of the two therapeutic agents may result in a superior depletion of serum testosterone available for binding to the androgen receptor in prostate cancer cells. The combination therapy may also provide an improved side effect profile, or allow for the use of lower dosages of androgen agonist.

Combination therapy may also be useful where patients are administered a dosage of androgen agonist sufficient to provide castrate levels of serum testosterone, and the disease has progressed to an androgen refractory stage. In this situation, it is proposed that while serum testosterone levels are decreased to very low levels, androgen present within the prostate cancer cell is still capable of fuelling growth of the tumor. Given that the aim of this therapy is to decrease the level of biologically available androgen within the cancer cell, it will be advantageous for the polypeptide to have the ability to enter the cell cytoplasm.

In addition, some prostate cancer epithelial cells might also secrete testosterone which is taken up by surrounding prostate cancer epithelial cells and our polypeptide drug would be able to soak up this source of androgen, irrespective of whether the polypeptide drug is able postal enter a prostate cancer epithelial cell directly.

In one form of the invention, the method of treatment or prevention includes administrates of a polypeptide of the present invention in combination with at least one other chemotherapeutic drug useful in the treatment of prostate cancer. Suitable compounds include, but are not limited to a cytostatic agent or cytotoxic agent. Nonlimiting examples of cytostatic agents are selected from: (I) microtubule-stabilizing agents such as but not limited to taxanes, paclitaxel, docetaxel; epothilones and laulimalides; (2) kinase inhibitors, illustrative examples of which include Iressa®, Gleevec, Tarceva™, (Erlotinib HCl), BAY-43-9006, inhibitors of the split kinase domain receptor tyrosine kinase subgroup (for example, 15 PTK787/ZK 222584 and SU11248); (3) receptor kinase targeted antibodies, which include, but are not limited to, Trastuzumab (HerceptinR), Cetuximab (Erbitux®), Bevacizumab (Avastin™), Rituximab (Ritusan®) Pertuzumab (Omnitarg™); (4) mTOR pathway inhibitors, illustrative examples of which include rapamycin and CCI-778; (5) Apo2L/Trail, antiangiogenic agents such as but not limited to endostatin, combrestatin, angiostatin, 20 thrombospondin and vascular endothelial growth inhibitor (VEGI); (6) antineoplastic immunotherapy vaccines, representative examples of which include activated T-cells, non-specific immune boosting agents (i.e., interferons, interleukins); (7) antibiotic cytotoxic agents such as but not limited to doxorubicin, bleomycin, dactinomycin, daunorubicin, epirubicin, mitomycin and mitozantrone; (8) alkylating agents, illustrative examples of which include Melphalan, Carmustine, Lomustine, Cyclophosphamide, Ifosfamide, Chlorambucil, Fotemustine, Busulfan, Temozolomide and Thiotepa; (9) hormonal antineoplastic agents, nonlimiting examples of which include Nilutamide, Cyproterone acetate, Anastrozole, Exemestane, Tamoxifen, Raloxifene, Bicalutamide, Aminoglutethimide, Leuprorelin acetate, Toremifene citrate, Letrozole, Flutamide, Megestrol acetate and Goserelin acetate; (10) gonadal hormones such as but not limited to Cyproterone acetate and Medoxyprogesterone acetate; (11) antimetabolites, illustrative examples of which include Cytarabine, Fluorouracil, Gemcitabine, Topotecan, Hydroxyurea, Thioguanine, Methotrexate, Colaspase, Raltitrexed and Capicitabine; (12) anabolic agents, such as but not limited to, Nandrolone; (13) adrenal steroid hormones, illustrative examples of which include Methylprednisolone acetate, Dexamethasone, Hydrocortisone, Prednisolone and Prednisone; (14) neoplastic agents such as but not limited to Irinotecan, Carboplatin, Cisplatin, Oxaliplatin, Etoposide and Dacarbazine; and (15) topoisomerase inhibitors, illustrative examples of which include topotecan and irinotecan.

In some embodiments, the cytostatic agent is a nucleic acid molecule, suitably an antisense or siRNA recombinant nucleic acid molecule. In other embodiments, the cytostatic agent is a peptide or polypeptide. In still other embodiments, the cytostatic agent is a small molecule. The cytostatic agent may be a cytotoxic agent that is suitably modified to enhance uptake or delivery of the agent. Non-limiting examples of such modified cytotoxic agents include, but are not limited to, pegylated or albumin-labeled cytotoxic drugs.

In specific embodiments, the cytostatic agent is a microtubule stabilizing agent, especially a taxane and preferably docetaxel. In some embodiments, the cytotoxic agent is selected from the anthracyclines such as idarubicin, doxorubicin, epirubicin, daunorubicin and mitozantrone, CMF agents such as cyclophosphamide, methotrexate and 5-fluorouracil or other cytotoxic agents such as cisplatin, carboplatin, bleomycin, topotecan, irinotecan, melphalan, chlorambucil, vincristine, vinblastine and mitomycin-C.

Illustrative agents for chemical hormone ablation therapy include GnRH agonists or antagonists such as Cetrorelix, agents that interfere with the androgen receptor including non-steroidal agents such as Bicalutamide and steroidal agents such as Cyproterone, and agents that interfere with steroid biosynthesis such as Ketoconazole. Chemical agents suitable for use in combination with the polypeptide and pharmaceutically acceptable salts as hormone ablation therapy for prostate cancer include, but are not limited to, non-steroidal anti-androgens such as Nilutamide, Bicalutamide and flutamide; GnRH agonists such as Goserelin acetate, leuprorelin and triptorelin; 5-alpha reductase inhibitors such as finasteride; and cyproterone acetate.

Given that the polypeptides of the present invention are proposed to be capable of decreasing the levels of biologically available androgen in the serum and/or in the prostate cancer cell, the combination therapy may provide an additive or synergistic effect.

In another aspect, the present invention provides a method for treating or preventing prostate cancer, the method comprising administering to a subject in need thereof an effective amount of a nucleic acid molecule or vector encoding a polypeptide as disclosed herein. The present invention encompasses the use of nucleic acids encoding the polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.” For gene therapy procedures in the treatment or prevention of human disease, see for example, Van Brunt (1998) Biotechnology 6:1 149 1154, the contents of which is incorporated herein by reference. Methods of treatment or prevention including the aforementioned nucleic acid molecules and vectors may include treatment with other compounds useful in the treatment of prostate cancer. Suitable compounds include, but are not limited to those described supra.

In a further aspect, the present invention provides a method for treating or preventing testosterone flare comprising administering to a subject in need thereof an effective amount of a polypeptide as described herein. LHRH drugs eventually result in suppression of testosterone, however before this occurs production of testosterone actually increases for a period. During the first week of treatment with a LHRH agonist or antagonist, the vastly increased production of testosterone may cause the cancer to flare.

In yet a further aspect, the present invention provides the use of a polypeptide as described herein in the manufacture of a medicament for the treatment or prevention of prostate cancer or testosterone flare.

In another aspect, the present invention provides the use of a nucleic acid molecule as described herein in the manufacture of a medicament for the treatment or prevention of prostate cancer or testosterone flare.

Still a further aspect provides the use of a vector as described herein in the manufacture of a medicament for the treatment of prevention of prostate cancer or testosterone flare.

The present invention will now be more fully described by reference to the following non-limiting Examples.

### EXAMPLES

### Example 1

### Construction of Androgen-Binding Polypeptide

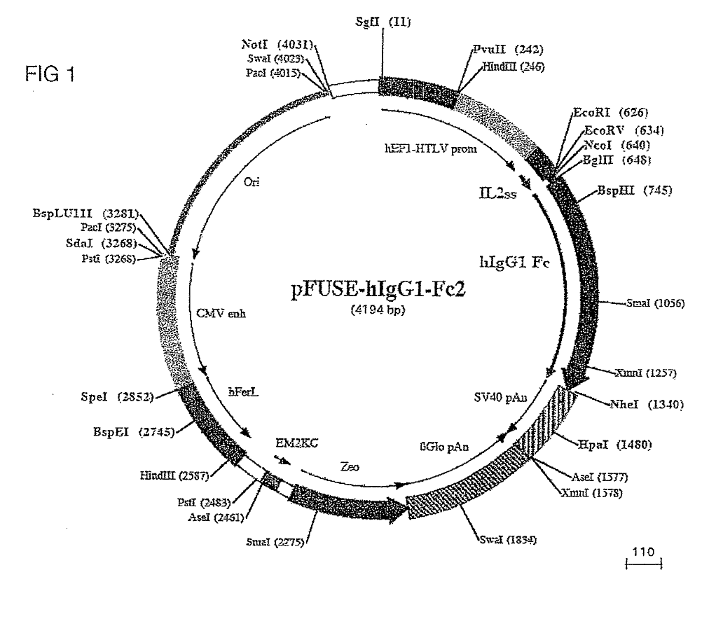

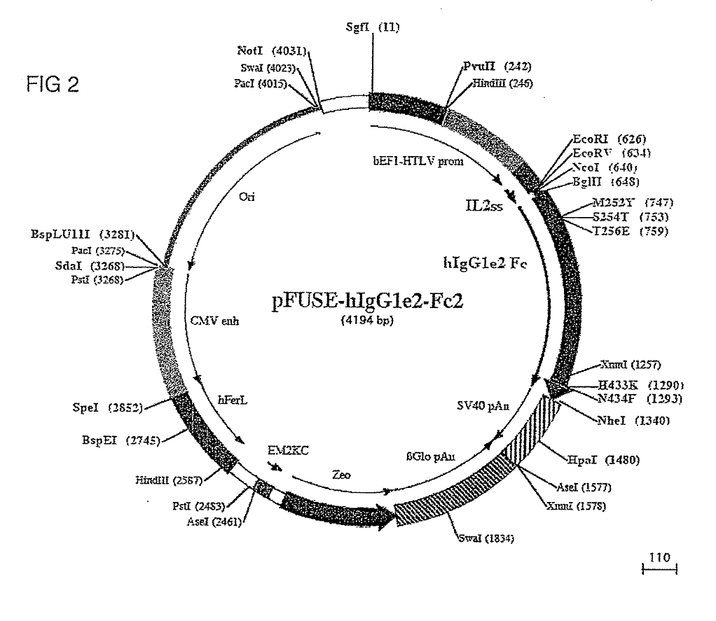

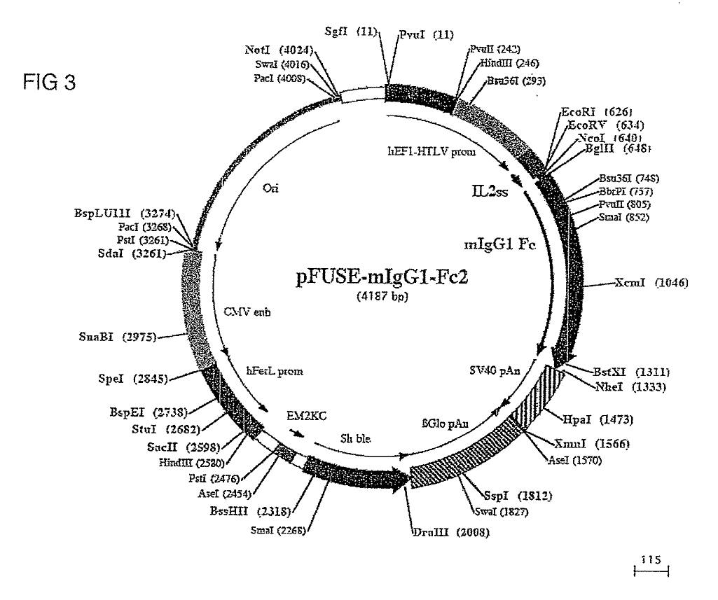

The following coding region (SEQ ID NO: 4) for human androgen receptor ligand binding domain (690 bp) is subcloned into various vectors (pFUSE-hlgG1-Fc2, pFUSE-hlgG1e2-Fc2, pFUSE-mlgG1-Fc2 from Invivogen) using EcoRI and BgIII RE sites (see FIGS. 1 to 3).

gacaacaaccagcccgacagcttcgccgccctgctgtccagcctgaacga
gctgggcgagaggcagctggtgcacgtggtgaagtgggccaaggccctgc
ccggcttcagaaacctgcacgtggacgaccagatggccgtgatccagtac
agctggatgggcctgatggtgttcgctatgggctggcggagcttcaccaa
cgtgaacagcaggatgctgtacttcgcccccgacctggtgttcaacgagt
acaggatgcacaagagcaggatgtacagccagtgcgtgaggatgaggcac
ctgagccaggaatttggctggctgcagatcaccccccaggaatttctgtg
catgaaggccctgctgctgttcagcatcatccccgtggacggcctgaaga
accagaagttcttcgacgagctgcggatgaactacatcaaagagctggac
aggatcatcgcctgcaagaggaagaaccccacctcctgcagcagaaggtt
ctaccagctgaccaagctgctggacagcgtgcagcccatcgccagagagc
tgcaccagttcaccttcgacctgctgatcaagagccacatggtgtccgtg
gacttccccgagatgatggccgagatcatcagcgtgcaggtgcccaagat
cctgagcggcaaggtcaagcccatctacttccacacccag

This sequence encodes the 230 C-terminal residues of the human androgen receptor protein disclosed herein as SEQ ID NO: 1.

The various vectors were separately transfected into CHO cells and secreted protein collected. The cell culture supernatant after various times of incubation was spun at 10,000-13,000 rpm for 15 min at 4° C. and filtered/concentrated prior to use.

### Cell Line

Mammalian CHO cell cultures were maintained in a Forme Scientific Incubator with 10% carbon dioxide at 37° C. in Dulbecco's Modified Eagle Medium (DMEM) (Gibco). Penicillin (100 U/ml), streptomycin (100 μg/ml) and amphotericin B (25 ng/ml) (Gibco Invitrogen #15240-062) were added to media as standard. As a routine, cells were maintained in the presence of 5% or 10% fetal bovine serum (Gibco Invitrogen #10099-141) unless otherwise stated. Subconfluent cells were passaged with 0.5% trypsin-EDTA (Gibco Invitrogen #15400-054).

### Propagation of DNA Constructs

DNA expression constructs were propagated in supercompetent DH5α E. Coli (Stratagene). To transform bacteria, 1 μg of plasmid DNA was added to 200 μl of bacteria in a microfuge tube and placed on ice for 20 mil. Bacteria were heat shocked at 42° C. for 1.5 min, then replaced on ice for a further 5 min. 1 ml of Luria-Bertani broth (LB) without antibiotics was then added, and the bacteria incubated at 37° C. on a heat block for 1 h. This was than added to 200 ml of LB with penicillin 50 μg/ml and incubated overnight at 37° C. with agitation in a Bioline Shaker (Edwards Instrument Company, Australia). The following morning the bacterial broth were transferred to a large centrifuge tube and spun at 10,000 rpm for 15 min. The supernatant was removed and the pellet dried by inverting the tube on blotting paper. Plasmid DNA was then recovered using the Wizard® Plus Midipreps DNA purification system (Promega #A7640). The pellet was resuspended in 3 ml of Cell Resuspension Solution (50 mM Tris-HCl pH 7.5, 10 mM EDTA₁ 100 μg/ml RNase A) and an equal volume of Cell Lysis Solution added (0.2 M NaOH₁ 1% SDS). This was mixed by inversion four times. 3 ml of neutralization solution (1.32 M potassium acetate pH 4.8) then added, and the solution again mixed by inversion. This was centrifuged at 14,000 g for 15 min at 4° C. The supernatant was then carefully decanted to a new tube by straining through muslin cloth. 10 ml of resuspended DNA purification resin was added to the DNA solution and mixed thoroughly. The Midi column tip was inserted into a vacuum pump, the DNA solution/resin mixture added to the column, and the vacuum applied. Once the solution was passed through the column it was washed twice by adding 15 ml of Column Wash Solution and applying the vacuum until the solution had drawn through. After the last wash the column was sharply incised to isolate the column reservoir which was transferred to a microfuge tube and spun at 13,000 rpm for 2 min to remove any residual wash solution. 100 μl of preheated nuclease-free water was added and the DNA eluted by centrifuging at 13,000 rpm for 20 sec in a fresh tube. DNA concentration was measured by absorbance spectroscopy (Perkin Elmer MBA2000).

### Examination of DNA Products by Gel Electrophoresis

The DNA products of polymerase chain reactions or restriction enzyme digests of plasmid DNA were analysed by agarose gel electrophoresis. Agarose (1-1.2%) was dissolved in TAE buffer (40 mM Tris acetate, 2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol was added to the samples before electrophoresis. Electrophoresis was conducted at approximately 100V in 1×TAE. DNA samples were visualized under ultraviolet light (254 nm).

### Polypeptide Fusion Protein Transfection and Expression in CHO Cells

The pFUSE-AR-hlgG1e2-Fc2 plasmid encoding the AR-LBD-IgG1 FC polypeptide fusion protein was transfected into CHO cells (ATCC) using Fugene HD (Roche, Cat No: 04709691001) and selected with Zeocin (Invitrogen, Cat No:R250-01). 2-5×10⁶ cells were then grown in 100-250 ml CHO-S-SFM II serum free suspension medium (Invitrogen, Cat No:12052-062) for 4-7 days. The cell culture was spun and the supernatant concentrated (using Amicon Ultra 15-50 kDa concentrators, Millipore Cat No:UFC905024).

### Analysis of Fusion Protein Expression Levels

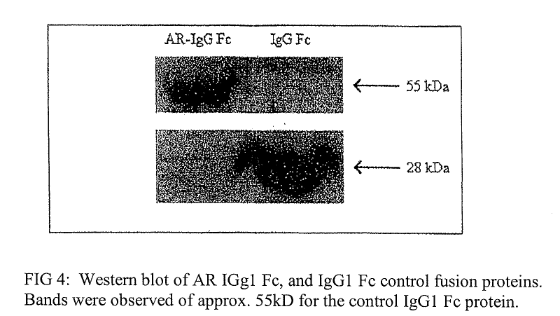

8 μl of concentrated AR or ER-LBD IgG Fc supernatant concentrates and 1 μl of concentrated IgG Fc control supernatants were loaded on to a 12% SDS page gel, and run at 170V for 70 min. The electrophoresed proteins were transferred on to nitrocellulose (100V for 90 min) using standard techniques. The nitrocellulose membranes were then probed with an Anti-Hu IgG Fc-HRP conjugate (Pierce, cat no:31413) at 1:20,000 dilution and developed using the Super Signal West Femto developing kit (Pierce, Cat No: 34094) according to the manufacturers specifications. The results are depicted in FIG. 4.

Clear expression of a single predominant polypeptide of size approx 55 kD was observed for both a AR-IgG1 Fc fusion protein as well as a ER-IgG1 Fc fusion protein. The control IgG1 Fc control protein of the correct size (28 kD) was also clearly apparent (FIG. 4).

### Example 2

### Efficacy of Polypeptide by In Vitro Assay

A human hormone sensitive prostate cancer cell line LNCaP₁ was exposed to the AR-LBD-IgG1 FC fusion protein as described in Example 1. The effects of the polypeptide on the growth and proliferation of the cells was then assessed.

As a control for hormone ablation therapy, the cells were cultured in hormone depleted serum (Charcoal stripped serum, CSS) as well as in normal serum to demonstrate growth in normal levels of androgens. In addition, LNCaP cells were also cultured in the presence of the non-steroidal antiandrogen nilutamide

### Cell Culture

The human prostate cancer cell line, LNCaP was obtained from American Type Tissue Collection (ATCC) and was routinely cultured in growth medium containing phenol red RPMI 1640 (Invitrogen, Auckland, New Zealand) supplemented with 10% fetal bovine serum (FBS₁ GIBCO) and 1 antibiotic/antimycotic mixture (Invitrogen, Auckland, New Zealand). Cells were maintained at 37° C. in 5% CO₂.

### In Vitro—Growth Proliferation Study.

2×10³ LNCaP cells were plated per well in a Falcon 96-well plate in 5% CO₂/37° C. in growth medium in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells were treated with either AR-LBD IgG1 Fc fusion protein (12 ng/ml) or IgG1 Fc control protein (12 ng/ml). In addition as control, 6 wells were treated with the nonsteroidal antiandrogen nilutamide (0.1 μM) as well as 6 wells with 10% charcoal stripped serum, to simulate steroid free conditions. After 120 hours in culture, cells were washed once with PBS and labeled with calcein (C1430, Molecular Probes, Oregon, USA) at 1 mM final concentration in PBS. Calcein positive cells were detected using a FLUOstar OPTIMA plate reader (BMG Labtech, Victoria, Australia). Experiments were performed in fc> replicates for each treatment condition.

### Statistical Analysis

Data are presented as mean±SEM unless otherwise indicated.

### Results

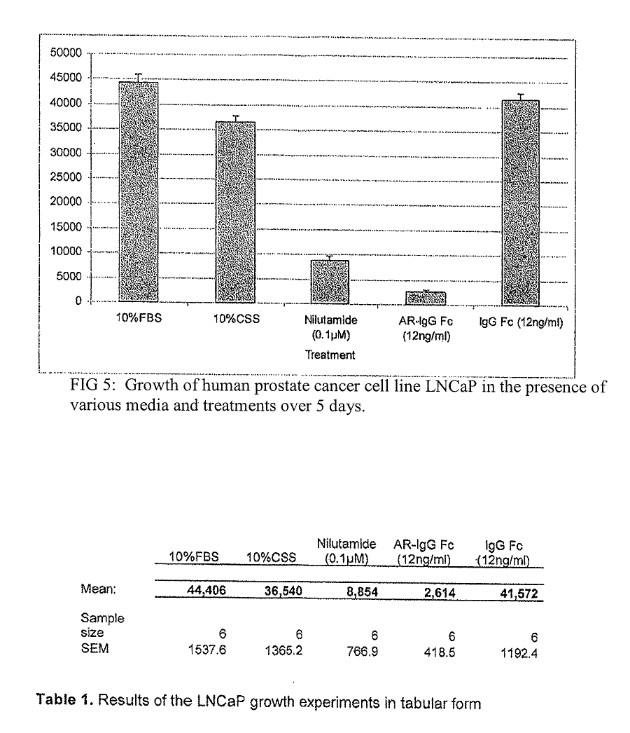

Treatment of the human hormone sensitive prostate cancer LNCaP cells with the AR IgG1 Fc fusion protein produced a dramatic effect on growth after 5 days exposure as assessed by the fluorescent calcein uptake assay. A 94% reduction in viable LNCaP cells was observed in wells treated with the AR IgG1 Fc fusion protein compared to LNCaP cells grown in media with complete 10% serum (FBS) (FIG. 5, Table 1). In comparison, the control IgG1 Fc protein lacking the AR LBD region had only a negligible effect on growth of the LNCaP cells with only a 6% decline in total cell number (FIG. 5, Table 1), indicating that the growth suppression effect is mediated via the androgen binding domain of the fusion protein. Growth of the LNCaP cells in media devoid of steroids, in the charcoal stripped serum (CSS) had only a modest effect on reducing LNCaP cell proliferation in the assay time frame, with a 18% decline observed (FIG. 5, Table 1). Interestingly, the AR IgG1 Fc fusion protein showed superior efficacy to the antiandrogen nilutamide in reducing LNCaP cell proliferation, with nilutamide reducing prostate cancer cell proliferation by 80% (FIG. 5, Table 1)

These results indicate that the AR IgG1 Fc fusion protein is able to suppress androgen mediated growth of prostate cancer cells. However, this suppression is occurring not only via depleting free androgen levels in the exogenous media, as growth of the LNCaP cells in media totally devoid of steroids had only a modest effect on the cellular proliferation. This superior effect of the AR IgG1 Fc protein compared to growth in steroid stripped serum indicates that the fusion protein is able to sequester endogenous androgens either internally or externally produced by the LNCaP cells.

### Example 3

### Efficacy of Polypeptide by In Vivo Assay. Rapid Reduction in Circulating Free Testosterone Levels

Athymic balb/c nude male mice, 6 weeks of age, were purchased from the Animal Resources Centre, Perth, Western Australia, and housed in a microisolator. Mice were given free access to standard rodent chow and drinking water throughout all experiments.

5 animals were administered IV tail vein injections of the AR-LBD IgG1 Fc fusion protein (25 ng in 200 μl of PBS). Three hours after injection the blood of all 5 mice was collected/pooled via mandibular bleeds (approx 100 μL blood per animal) in Lithium/heparin tubes. In addition, 5 control athymic balb/c nude male mice of the same sex and age were similarly bled at the same time and samples pooled. The unclotted blood was then spun at 2500 rpm for 5 min to separate the red blood cells from the serum. 100 μl samples of pooled serum were then run according to the manufacturers specification of the Coat-a-count Free testosterone kit (Siemens, Cat No: TKTF1).

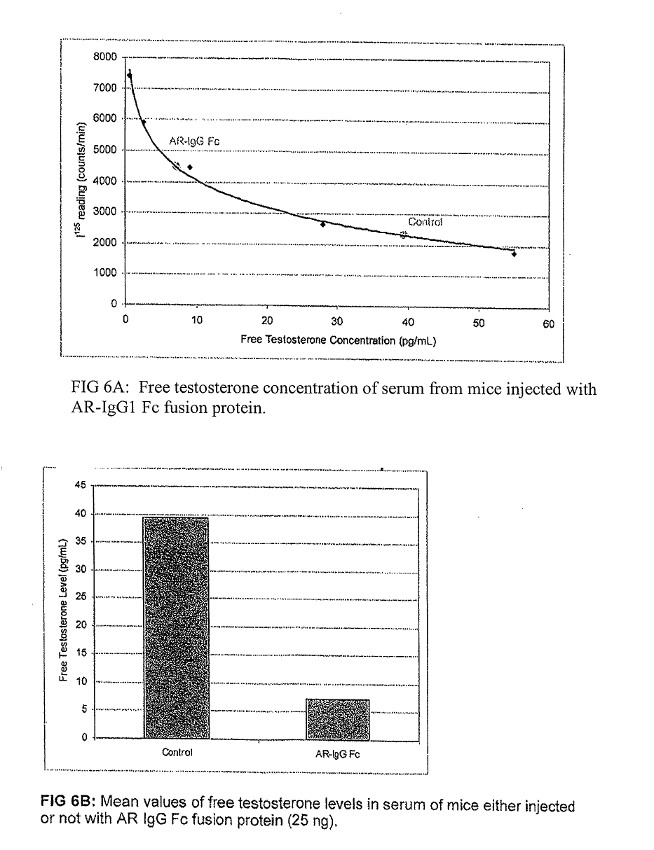

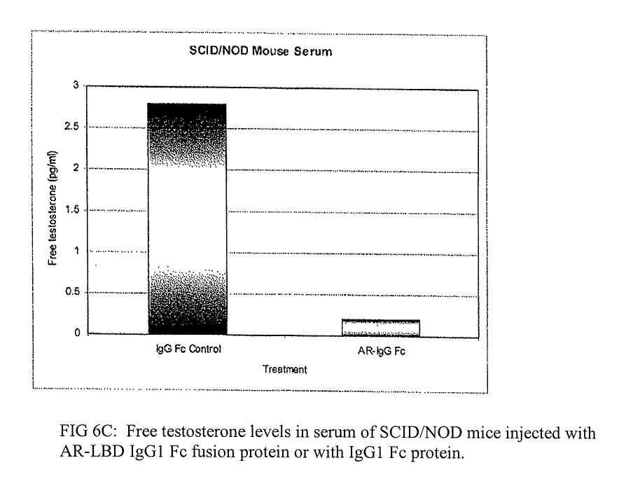

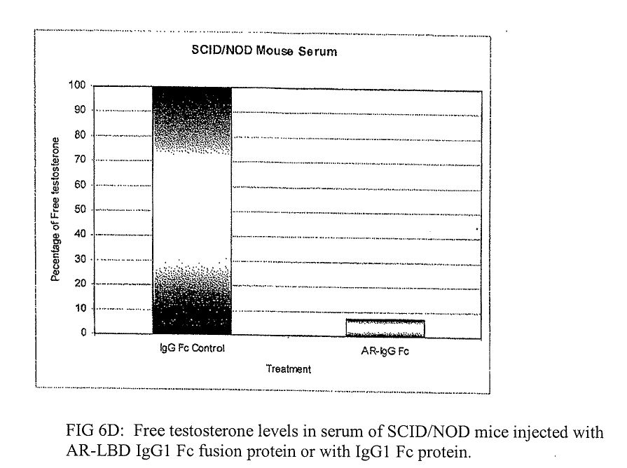

The results are depicted in FIG. 6A, B and Table 2. The free testosterone levels in the serum of the control mice averaged 39.44 pg/ml. However, the free testosterone levels of the mice injected with the AR IgG1 Fc fusion protein was only 7.23 pg/ml. This represents a dramatic 82% decline in bioavailable testosterone levels in only 3 hours after injection.

In a further experiment, 6 SCID/NOD male mice, 5 weeks of age were purchased from the Animal Resources Centre, Perth, Western Australia, and housed in a microisolator. Mice were given free access to standard rodent chow and drinking water throughout all experiments. The animals were then separated into two groups of 3 mice. Three animals in one group were administered IV tail vein injections of the AR-LBD IgGI Fc fusion protein (200 μl of 1 ng/μl of PBS). Three mice in the other control group were then administered IV tail vein injections of the control IgG1 Fc protein (200 μl of 1 ng/μl of PBS). Four hours after injection the blood of all 6 mice was collected via mandibular bleeds (approx 100 blood per animal) in Lithium/heparin tubes. The unclotted blood was then spun at 2500 rpm for 5 min to separate the red blood cells from the serum. 100 μl samples of pooled serum were then run according to the manufacturers specification of the Coat-a-count Free testosterone kit (Siemens, Cat No: TKTFI).

The results are depicted in FIGS. 6C and D. The free testosterone levels in the serum of the control mice injected with the control IgG1 Fc protein averaged 2.8 pg/ml. However, the free testosterone levels of the mice injected with the AR-LBD IgG1 Fc fusion protein was only 0.2 pg/ml. This represents a dramatic 93% decline in bioavailable testosterone levels only 4 flours after injection.

### Example 4

### Efficacy of Polypeptide by In Vivo Assay

A xenograft animal model of an androgen dependent tumor is used to assess efficacy in vivo. 5-7 week old SCID (severe combined immunodeficiency) or athymic balb/c nude male mice are purchased from the Animal Resources Centre, Perth, Western Australia, and housed in microisolators. Mice are given free access to standard rodent chow and drinking water throughout all experiments.

### Subcutaneous Tumor Models

To establish flank prostate tumours, 4×105 washed LNCaP cells were resuspended in 50 μl PBS, mixed with an equal volume of Matrigel (BD #354234) and injected subcutaneously into the right flank of 6 week old male nude mice with a 23G needle. Following tumour cell injection, 100 μl of 1 ng/μl control IgG1 Fc was injected into the flanks of three mice and 100 μl of 1 ng/μl AR-LBD IgG1 Fc fusion protein injected into the flanks of the three remaining mice. Seven days later, a second flank injection of 200 μl of 1 ng/μ| IgG 1 Fc was administered to the three animals in the control group and 200 μl of 1 ng/μ| AR-LBD IgGI Fc fusion protein was administered to the three animals in the active treatment group. No further treatment was given and the animals were monitored and tumour sizes measured regularly. The experiment was terminated 5 weeks after the initial tumour cell injection, and final tumour volumes and weight were recorded.

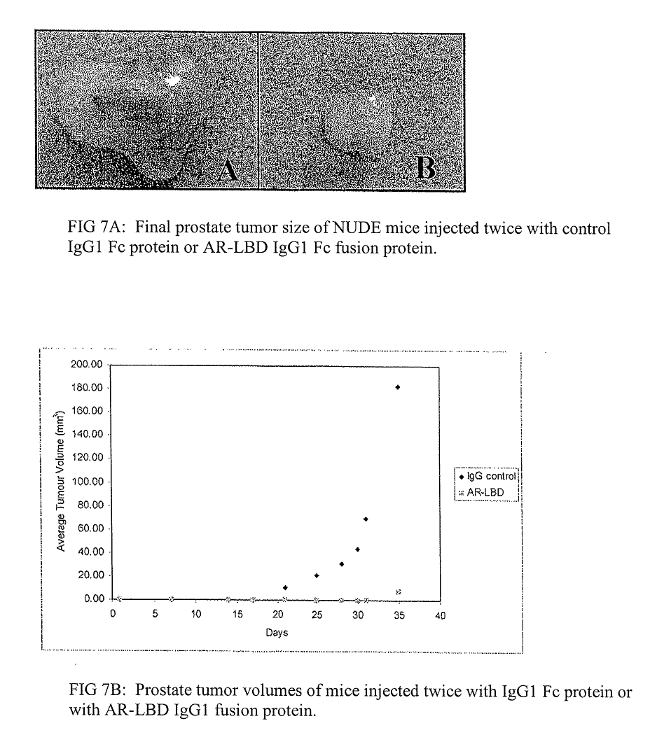

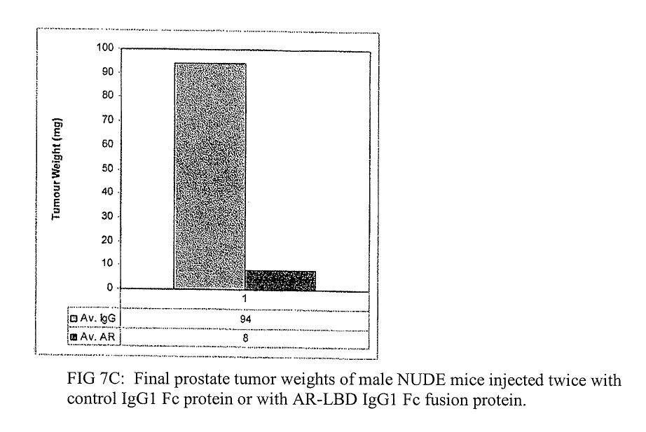

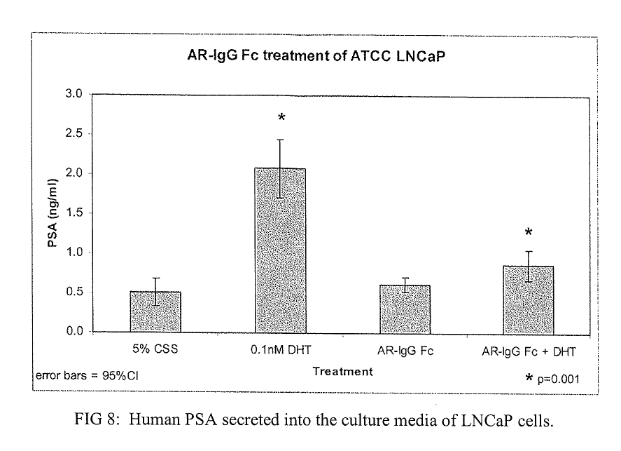

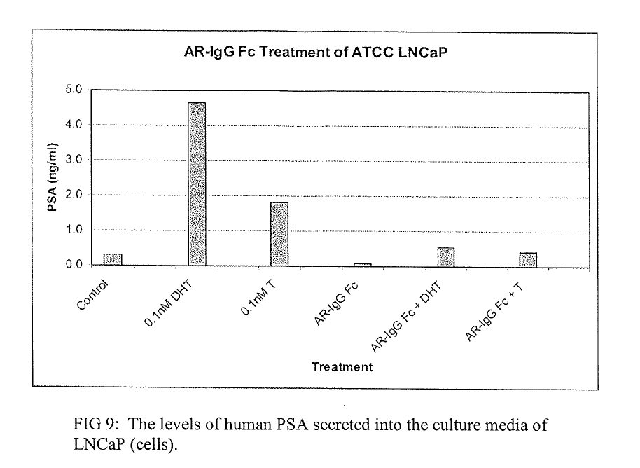

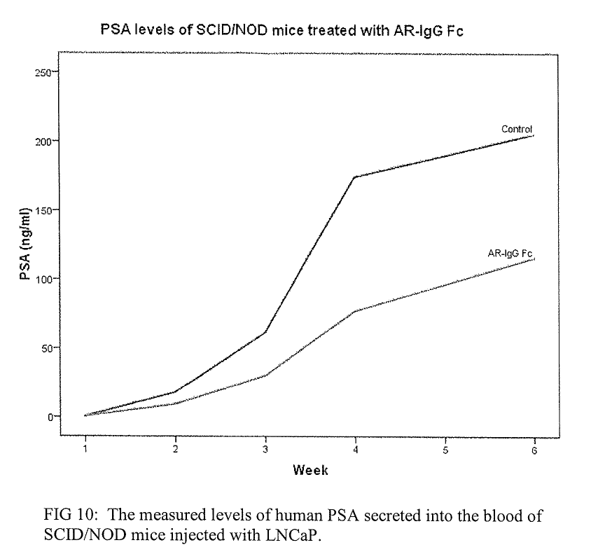

The results are depicted in FIGS. 7A, B and C. The final tumour volume of the control mice injected with the IgG1 Fc protein averaged 182.9 mm3. However, the final tumour volume of the mice injected with the AR-LBD IgG1 Fc fusion protein was only 7.3 mm3 (FIGS. 7A and B). There was also a significant effect of the AR-LBD IgGI Fc fusion protein in inhibiting prostate tumour growth throughout the experiment with animals treated with the androgen binding fusion protein only developing very small tumours at the end of the experiment (FIG. 7B). This was in marked contrast with animals injected with the control IgG1 protein which developed tumours much earlier and which were much larger at the end of the experiment (FIG. 7B).

There was similarly a very large effect of the AR-LBD IgG1 Fc fusion protein on final tumour weights with average weight being only 8 mg whilst control mice injected with the IgG1 Fc protein averaged 94 mg (FIG. 7C).

### Orthotopic Model of Hormone Dependent Prostate Cancer

Orthotopic tumours are established as follows. Mice (between 6-10 per treatment group) are anaesthetized with a mixture of ketamine 100 mg/kg and xylazine 20 mg/kg injected to allow a small transverse lower abdominal incision to be made. The bladder, seminal vesicles and prostate are 5 delivered into the wound and 1×10⁶ LNCaP cells in 20 μl of cell culture medium with Matrigel injected into the dorsolateral prostate with a 29 gauge needle. Injections are performed with the aid of an operating microscope at ×10 magnification. A technically satisfactory injection is confirmed by the formation of a subcapsular bleb and the absence of visible leak. The lower urinary tract is replaced and the anterior abdominal wall closed with 4/0 silk. The skin is apposed with surgical staples. Postoperatively the animals are given an intraperitoneal injection of normal saline at a calculated volume of 3-5% of the pre-anaesthetic weight. Mice are recovered under radiant heating lamps until fully mobile.

Animals ate divided into treatment groups of 6-10 mice and after different time periods following tumour cell injection are administered IV tail vein injections of the polypeptide at different concentrations (optimised from in vitro experimental results). At the end of the experiment mice are sacrificed by 2.0 carbon dioxide narcosis. The prostate, seminal vesicles and bladder are removed en bloc, and appendages carefully dissected from the tumour containing prostate if not grossly involved. The tumour containing prostate gland is weighed, and diameter measured in three dimensions with Vernier calipers. The retroperitoneum is explored under magnification cephadally to the level of the renal veins. Lymph nodes found in the para-aortic and para-iliac areas are dissected free and their long axis measured. Tissue for Immunohistochemical staining is embedded in OCT and frozen in liquid nitrogen cooled isopentane. Tumours are stored at −70° C. until analysis.

### Surgical Castration

As controls for hormone ablation therapy, Mice are anaesthetized with a mixture of ketamine 100 mg/kg and xylazine 20 mg/kg injected intraperitoneally to allow a small transverse lower abdominal incision to be made. The lower genitourinary organs are delivered into the wound, the vas deferens and vascular pedicle ligated with 4/0 silk, the testes excised. The abdomen is dosed with 4/0 silk with clips to skin. Mice are recovered on a heating pad until fully recovered.

### Local Tumour Growth in Orthotopic Models of ADPC

At specified times post inoculation (from days 25-42), mice are euthanased by carbon monoxide narcosis and a necroscopy performed. The abdomen is opened in the midline from sternum to pubis and retracted, and the abdominal organs inspected. Under magnification, the urethra is transected at the prostatic apex and the ureters and vas deferentia are identified bilaterally and divided close to the prostate. The specimen is then removed en bloc and the seminal vesicles and bladder dissected free under magnification. The tumour containing prostate gland is then weighed and its dimensions measured in 3 axes with Vernier calipers. Where a discrete nodule is found this is dissected away and weighed separately.

After these measurements, the prostate or tumour is embedded in OCT, snap frozen in liquid nitrogen cooled isopentane and stored at −70° C. until use. Prostate glands without macroscopic tumours are serially sectioned and analysed histologically to confirm the presence of tumour.

Volume of the tumour containing prostate gland is calculated using the formula a*b*c, where a, b and c represent maximum length of the gland measured with Verniers calipers in three dimensions at right angles to one another.

### Example 5

### Safety and Efficacy of Polypeptide in Human Subjects

This Example is directed to patients with early hormone refractory prostate cancer (HRPC). While it would be possible (and desirable) to trial the polypeptide in patients with hormone dependent tumours, patients with HRPC are used at first instance for ethical reasons. HRPC patients have failed their first line hormone ablation therapy and have no other treatment options until they progress to metastases, when chemotherapy becomes an option. Furthermore, these patients have low levels of circulating testosterone (as they typically remain on androgen ablation therapy, but not on androgen antagonist drugs) and their PSA levels would be just starting to rise. This approach allows an assessment of whether the polypeptide is well tolerated, the effects on levels of biologically available testosterone levels, and also levels PSA.

### Objectives

The primary objectives of this study are to determine the safety and tolerability of intra venous infusions of the polypeptide binding protein in patients with HRPC, and to evaluate its pharmacokinetic profile when given as a single IV infusion once every three weeks. Secondary objectives include: to determine whether treatment with polypeptide binding protein can lead to clinical responses as determined by serum PSA in patients with HRPC; to estimate the duration of PSA response (decline); to estimate progression-free survival; to determine whether treatment with polypeptide binding protein can lead to biological responses in patients with HRPC; and to evaluate the PSA slope before and during polypeptide binding protein therapy.

### Study Design

This study describes an open label phase I dose escalation study. After signing informed consent, patients undergo baseline testing to confirm eligibility. Patients then commence treatment with polypeptide binding protein, administered as a single intravenous infusion once every three weeks (one cycle). After four cycles of therapy (12 weeks), patients with stable or responding disease, and who wish to continue on study, are offered treatment extension for up to another four cycles. All patients are assessed for safety 28 days after the last dose of study drug, and where possible, are evaluated three months after their final treatment of study drug. In total, 12-15 patients (4-patients per dose level) are recruited from a variety of multidisciplinary urooncology clinics.

### Patient Eligibility

Patients are screened for study eligibility based on the following inclusion and exclusion criteria.

To be eligible for enrolment, patients must fulfill the following criteria:
1. Provision of written informed consent
2. Male, aged 18 years or older
3. Hormone refractory prostate cancer confirmed by castrate serum testosterone levels and at least three elevated and rising PSA levels, with at least two weeks between measurements
4. The PSA level must be greater than 5 μg/l at study entry
5. Patients may be asymptomatic or have only minor symptoms due to prostate cancer
6. WHO performance status <2
7. Anti-androgen therapy must have been stopped at least 4 weeks before entry into the trial, with evidence of continuing PSA rises after this time. LHRH agonists or antagonists should be continued and are allowed concurrently
8. Life expectancy of at least six months
   Any of the following is regarded as a criterion for exclusion from the trial:
1. Prior cytotoxic chemotherapy for hormone refractory prostate cancer
2. Prior strontium therapy
3. Treatment with an investigational agent in the last 4 weeks
4. Other co-existing malignancies or malignancies diagnosed within the last 5 years with the exception of non-melanomatous skin cancer
5. Any unresolved chronic toxicity greater than CTC grade 2 from previous anticancer therapy
6. Incomplete healing from previous surgery
7. Absolute neutrophil counts <1×10⁹ Λ or platelets <100×10⁹
8. Serum bilirubin >1.25 times the upper limit of reference range (ULRR)
9. In the opinion of the investigator, any evidence of severe or uncontrolled systemic disease (e.g. unstable or uncompensated respiratory, cardiac, hepatic or renal disease)
10. Serum creatinine >1.5 times the ULRR
11. Alanine aminotransferase (ALT) or aspartate aminotransferase (AST)>2.5 times the ULRR
12. Evidence of any other significant clinical disorder or laboratory finding that makes it undesirable for the patient to participate in the trial
13. Patients may not use unapproved or herbal remedies for prostate cancer
14. A history of alcoholism, drug addiction, or any psychiatric condition which in the opinion of the investigator would impair the patient's ability to comply with study procedures.

### Study Agent

The polypeptide is produced in accordance with Example 1. All formulation and packing of the study agent is in accordance with applicable current Good Manufacturing Practice (GMP) for Investigation Medicinal Products as specified by the Therapeutic Goods Administration (Australia) and meet applicable criteria for use in humans.

### Treatment Plan

Three dose levels of polypeptide binding protein are investigated (0.3, 1.0, and 3.0 mg/kg). After enrollment in the 0.3-mg/kg cohort is complete, there is a 2-week waiting period before the 1.0-mg/kg cohort is begun. There is also a 2-week waiting period after the 1.0-mg/kg cohort is enrolled before enrollment of the 3.0-mg/kg cohort is begun.

Individual patient doses are prepared by diluting the appropriate volume of polypeptide binding protein (25 mg/ml) with 0.9% sodium chloride to yield a final concentration of 4 mg/ml. The volume of solution prepared is 25 to 150 ml, depending on the patient's dose and body weight. The polypeptide is infused over a period of no less than 1 hour by a registered nurse or physician's assistant under the guidance of one of the trial investigators. In addition, internists or anesthesiologists are present to oversee the administration of the study agent and aid in the management of adverse events.

All adverse events are graded according to the Common Terminology Criteria for Adverse Events Version 3.0 (Cancer Therapy Evaluation Program, DCTD, NCl, NIH, DHHS, Mar. 31, 2003, http://ctep.cancer.gov). DRT and DLT is based on the first three weeks of treatment. DRT is defined as any Grade 2 non-haematological or Grade 3 haematological toxicity. DLT is defined as any Grade 3/4 non-haematological or Grade 4 haematological toxicity. Patients who require other treatment for progressive prostate cancer, such as radiotherapy to new metastatic lesions, surgery or chemotherapy, are removed from the study and are not replaced. Treatment will not be administered if there is ≧Grade 2 haematological and/or non-haematological toxicity. Treatment may be re-initiated once the toxicity is <Grade 1, with treatment delayed for up to two weeks. In the absence of treatment delays, treatment may continue for up to four cycles or until there is disease progression; intercurrent illness prevents further administration of treatment; unacceptable adverse events occur; the patient decides to withdraw from the study; or general or specific changes in the patient's condition render the patients unacceptable for further treatment in the judgment of the trial investigator.

### Pre-Treatment and Treatment Evaluation

At study entry, patients are screened for measurable disease by radionuclide hone scintigraphy and computed tomography of the chest, abdomen and pelvis. In patients with measurable disease, tumour response is assessed according to the Response Evaluation Criteria in Solid Tumours (Therasse, P., et al., J Natl Cancer Inst, 2000. 92(3): p. 205-1 B). Given the stage of disease at which patients are enrolled, it is anticipated that the majority will not have measurable disease at the time of study entry. However, patients will have a rising PSA, which is measured every three weeks for the duration of the study. Therefore in patients with no radiologically evaluable disease, PSA response is used as a surrogate marker of tumour response, defined as a reduction in PSA of at least 50% below the level measured at study entry, documented on at least two separate occasions at least four weeks apart. PSA progression is defined as the time from the first PSA decline ≦50% of baseline until an increase in PSA above that level. Toxicity is evaluated according to the Common Terminology Criteria for Adverse Events Version 3.0.

### Sample Collection

Sample collection to determine population pharmacokinetic parameters for polypeptide binding protein is performed in patients accrued to the study. Serial blood samples (10 ml/sample) are collected at the following times: pre-dose (within 60 min prior to study drug administration) and post-dose at 30 min, 1 , 2, 4, 6, 24, 48 and 72 h. In addition, trough samples are taken at days 7, 14 and 21, weeks. Blood samples are collected into heparinised vacutainers for assessment of sodium selenate status. The plasma is separated by centrifugation (2000 g at 4° C. for 15 min). Following centrifugation, the plasma is separated into three aliquots (each approximately 1 ml) and placed in identically labeled polypropylene tubes. Samples are frozen at −80° C. until analysis.

### Study Completion

A patient is considered to have completed the study following the evaluations for the primary endpoint after 4 cycles of treatment. However, patients continuing on study and receiving further treatment are followed and data collected. Where possible, all patients are evaluated every three months. The study is closed when the final patient has undergone this last review. Patients who have received at least 1 cycle of study agent are evaluable for safety and for clinical and biological response. PSA response rates are summarised by proportions together with 95% confidence intervals. Proportions and durations of progression-free survival are summarised by Kaplan-Meier methods. Toxicity is summarised according to Common Terminology Criteria for Adverse Events Version 3.0.

### Example 6

### Efficacy of Androgen-Binding Polypeptide by In Vitro Assay. Measurement of Secreted PSA Levels in a Hormone Dependent Prostate Cancer Cell Line

A human hormone sensitive prostate cancer cell line, LNCaP (ATCC), was exposed to a polypeptide as described in Example 1. The effects of the polypeptide on the production and secretion of the prostate specific antigen, PSA, was then assessed.

As a control for hormone ablation therapy, the cells were cultured in hormone depleted serum (Charcoal stripped serum, CSS) to demonstrate effect of androgen depletion on PSA production and secretion.

### Cell Culture.

The human prostate cancer cell line, LNCaP was obtained from American Type Tissue Collection (ATCC) and was routinely cultured in growth medium containing phenol red RPMI 1640 (Invitrogen, Auckland, New Zealand) supplemented with 10% fetal bovine serum (FBS, 15 GIBCO) and 1% antibiotic/antimycotic mixture (Invitrogen, Auckland, New Zealand). Cells were maintained at 37° C. in 5% CO2. Dilutions were made for the polypeptide (20 ng/ml) in 5% charcoal strip serum (CSS, HyClone) for in vitro experiments.

### In Vitro—PSA Production and Secretion Study.

3×10³ LNCaP cells were plated per well in quadruplicate in a Falcon 96-well plate and allowed to attach overnight at 5% CO2/37° C. in growth medium (as indicated above). The medium was replaced with fresh complete growth medium after 36 hours. Treatments comprising either 5% CSS alone, or 0.1 nm DHT (dihydrotestosterone), or 20 ng/ml AR-IgG Fc polypeptide, or polypeptide and DHT combination were then added to the wells in quadruplicate. After 120 hours in culture, cell culture media was collected from each well and a PSA reading was measured in duplicate for each well. Human PSA was measured according to the manufacturers methodology using the PSA ELISA from Diagnostic Systems Laboratories (cat number: DSL-10-9700).

### Statistical Analysis

Data are presented as means unless otherwise indicated. Error bars represent the 95% confidence intervals. Differences between treatment groups are analyzed using Fisher's least significant difference test with significance assumed at 99% confidence interval, for p>0.01, One-Way ANOVA. All statistical analysis was performed using STATGRAPHICS statistical software (Virginia, USA).

### Results

The results clearly indicate that the addition of 0.1 nM DHT is able to significantly boost production of secreted PSA in the media of the LNCaP cells when compared to levels of PSA production in the 5% CSS media cells alone, confirming that these cells are androgen sensitive and that production of PSA in these cells can be increased by exogenous DHT. Addition of the AR-IgG Fc polypeptide to the cells was able to significantly block the DHT 10 driven boost in PSA production in the LNCaP cells indicating that the polypeptide can efficiently inhibit the biological activity of an androgen.

### Example 7

### Efficacy of Androgen-Binding Polypeptide by In Vitro Assay. Inhibition of Androgen Activity as Assessed by PSA Production in a Hormone Dependent Cell Line

A human hormone sensitive prostate cancer cell line, LNCaP (ATCC), was exposed to different androgens and a polypeptide as described in Example 1. The effects of the polypeptide on the production and secretion of the prostate specific antigen, PSA, was then assessed.

As a control for hormone ablation therapy, the cells were cultured in hormone depleted serum (Charcoal stripped serum, CSS) to demonstrate effect of androgen depletion on PSA production and secretion.

### Cell Culture.

The human prostate cancer cell line, LNCaP was obtained from American Type Tissue Collection (ATCC) and was routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells were maintained at 37° C. in 5% CO2. Dilutions were made for the polypeptide (20 ng/ml) in 5% charcoal strip serum (CSS, HyClone) for in vitro experiments.

### In Vitro—PSA Production and Secretion Study.

3×10³ LNCaP cells were plated per well in quadruplicate in a Falcon 96-well plate and allowed to attach overnight at 5% CO2/37° C. in growth medium (as indicated above). The medium was replaced with fresh complete growth medium after 36 hours. Treatments comprising either 5% CSS alone, or 0.1 nm T (testosterone), or 0.1 nm DHT (dihydrotestosterone), or 20 ng/ml AR-IgG Fc polypeptide, or polypeptide and T or DHT combination were then added to the wells in sextuplet. After 120 hours in culture, cell culture media was collected from each well and a PSA reading was measured in duplicate for each well. Human PSA was measured according to the manufacturers methodology using the PSA ELISA from Diagnostic Systems Laboratories (cat number: DSL-10-9700).

### Statistical Analysis

Data are presented as means unless otherwise indicated.

### Results

The results clearly indicate that the addition of 0.1 nM DHT is able to strongly boost production of secreted PSA in the media of the LNCaP cells when compared to levels of PSA production in the 5% CSS media cells alone, confirming that these cells are androgen sensitive and that production of PSA in these cells can be increased by exogenous DHT. Likewise addition of 0.1 nM Testosterone to the cells (T) is also able to clearly increase production of PSA, though not as strongly as observed for DHT. Addition of the AR-IgG Fc polypeptide to the cells was able to significantly block both the DHT and T driven boost in PSA production in the LNCaP cells indicating that the polypeptide can efficiently inhibit the biological activity of either dihydrotestosterone or testosterone.

### Example 8

### Efficacy of Androgen-Binding Polypeptide by In Vivo Assay. Inhibition of PSA Production in an Orthotopic Model of Hormone Dependent Prostate Cancer

Orthotopic tumours were established as follows. Immunocompromised SCID/NOD mice (4-per treatment group) were anaesthetized with a mixture of ketamine 100 mg/kg and xylazine 20 mg/kg injected intraperitoneally to allow a small transverse lower abdominal incision to be made. The bladder, seminal vesicles and prostate were delivered into the wound and 5×10⁶ LNCaP cells in 20 μl of cell culture medium with Matrigel injected into the dorsolateral prostate with a 29 gauge needle. Injections were performed with the aid of an operating microscope at ×10 magnification. A technically satisfactory injection is confirmed by the formation of a subcapsular bleb and the absence of visible leak. The lower urinary tract was replaced and the anterior abdominal wall closed with 4/0 silk. The skin was apposed with surgical staples. Postoperatively the animals were given an intraperitoneal injection of normal saline at a calculated volume of 3-5% of the pre-anaesthetic weight. Mice recovered under radiant heating lamps until fully mobile.

Animals were divided into treatment groups of 4 mice and after 1 week following tumor implantation were administered 100 μl by IV tail vein injections of either a control IgG Fc or the AR-IgG Fc polypeptide (1-2 ug/ml). Weekly injections of the control or androgen binding polypeptide were then administered until the end of the experiment (6 weeks). Weekly blood sampling was performed in all mice by mandibular vein bleeding where typically 50-100 μl of blood was obtained and immediately spun and processed for PSA measurements. At the end of the experiment mice are sacrificed by carbon dioxide narcosis.

### Cell Culture.

The human prostate cancer cell line, LNCaP was obtained from American Type Tissue Collection (ATCC) and routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells were maintained at 37° C. in 5% CO2.

### Plasma PSA Determination.

Human PSA was measured according to the manufacturers methodology using the PSA ELISA from Diagnostic Systems Laboratories (cat number: DSL-10-9700).

### Statistical Analysis

Data are presented as means unless otherwise indicated.

### Results

The results clearly indicate that the treatment with the AR-IgG Fc polypeptide was able to clearly reduce PSA plasma levels in SCID mice compared to control animals injected with a IgG Fc polypeptide alone. This indicates that the AR-IgG Fc polypeptide can efficiently inhibit the biological activity of either circulating endogenous androgens.

Finally, it is to be understood that various other modifications and/or alterations may be made without departing from the spirit of the present invention as outlined herein.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

1-36. (canceled)

37. A polypeptide comprising an androgen binding region, the androgen binding region capable of binding to an androgen at a sufficient affinity or avidity such that upon administration of the polypeptide to a mammalian subject the level of biologically available androgen is decreased.

38. A polypeptide according to claim 37, wherein the level of biologically available androgen is measured in the blood of the subject, or a prostate cell of the subject, or a prostate cell.

39. A polypeptide according to claim 37, wherein the level of biologically available androgen is decreased such that the growth of a prostate cancer cell in the subject is decreased or substantially arrested.

40. A polypeptide according to claim 37, wherein the level of biologically available androgen is decreased such that the growth of a prostate cancer cell in the subject is decreased or substantially arrested.

41. A polypeptide according to claim 37, having an affinity for an androgen that is equal to or greater than the affinity between the androgen and a protein that naturally binds to the androgen.

42. A polypeptide according to claim 37, wherein where the androgen is testosterone, the protein that naturally binds to the androgen is selected from the group consisting of sex hormone binding globulin, the 5-alpha-reductase enzyme present in a prostate epithelial cell, and the androgen receptor present in a prostate epithelial cell.

43. A polypeptide according to claim 37, having an affinity for dihydrotestosterone that is equal to or greater than for the affinity between dihydrotestosterone and the androgen receptor present in a prostate epithelial cell.

44. A polypeptide according to claim 37, wherein the androgen binding region includes the androgen binding domain from the human androgen receptor, or the androgen binding domain from the sex hormone binding globulin.

45. A polypeptide according to claim 37, having a single androgen binding region.

46. A polypeptide according to claim 37, comprising a carrier region.

47. A polypeptide according to claim 46, wherein the carrier is the Fc region of human IgG.

48. A polypeptide according to claim 37, that is selected from the group consisting of a fusion protein, a monoclonal antibody, a polyclonal antibody, and a single chain antibody.

49. A polypeptide according to claim 37, comprising a multimerisation domain.

50. A nucleic acid molecule capable of encoding a polypeptide according to claim 37.

51. A vector comprising a nucleic acid molecule according to claim 50.

52. A composition comprising a polypeptide according to claim 37, and a pharmaceutically acceptable carrier.

53. A method for treating or preventing prostate cancer or testosterone flare in a subject, the method comprising administering to a subject in need thereof an effective amount of a ligand capable of binding androgen in the subject, such that the level of biologically available androgen in the subject is decreased as compared with the level of biologically available androgen present in the subject prior to administration of the polypeptide.

54. A method according to claim 53, wherein the ligand is a polypeptide according to claim 37.

55. A method for treating or preventing prostate cancer or testosterone flare in a subject, the method comprising administering to a subject in need thereof an effective amount of a polypeptide according to claim 37, or a nucleic acid molecule according to claim 37, or a vector according to claim 51.

56. A method according to claim 53, wherein the prostate cancer is in the androgen dependent phase.


SEQUENCE LISTING (4 sequences)

SEQ ID NO: 1 - protein, 919 aa, Homo sapiens
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 2 - protein, 401 aa, Homo sapiens
ESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 3 - protein, 224 aa, Homo sapiens
THTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 690 bp, Homo sapiens
gacaacaaccagcccgacagcttcgccgccctgctgtccagcctgaacgagctgggcgagaggcagctggtgcacgtggtgaagtgggccaaggccctgcccggcttcagaaacctgcacgtggacgaccagatggccgtgatccagtacagctggatgggcctgatggtgttcgctatgggctggcggagcttcaccaacgtgaacagcaggatgctgtacttcgcccccgacctggtgttcaacgagtacaggatgcacaagagcaggatgtacagccagtgcgtgaggatgaggcacctgagccaggaatttggctggctgcagatcaccccccaggaatttctgtgcatgaaggccctgctgctgttcagcatcatccccgtggacggcctgaagaaccagaagttcttcgacgagctgcggatgaactacatcaaagagctggacaggatcatcgcctgcaagaggaagaaccccacctcctgcagcagaaggttctaccagctgaccaagctgctggacagcgtgcagcccatcgccagagagctgcaccagttcaccttcgacctgctgatcaagagccacatggtgtccgtggacttccccgagatgatggccgagatcatcagcgtgcaggtgcccaagatcctgagcggcaaggtcaagcccatctacttccacacccag